(19) United States
(12) Patent Application Publication
(10) Pub. No.: US 2020/0251780 A1
(43) Pub. Date: Aug. 6, 2020

(54) ELECTROLYTE, LITHIUM BATTERY INCLUDING THE SAME, AND METHOD OF MANUFACTURING THE LITHIUM BATTERY

(71) Applicants: Samsung Electronics Co., Ltd., Suwon-si, (KR); Samsung SDI Co., Ltd., Yongin-si, (KR)

(72) Inventors: Insun PARK, Suwon-si (KR); Myongchun KOH, Hwaseong-si, (KR); Dongyoung KIM, Yongin-si, (KR); Jinah SEO, Seoul (KR)

(21) Appl. No.: 16/778,003

(22) Filed: Jan. 31, 2020

(30) Foreign Application Priority Data
Feb. 1, 2019 (KR) 10-2019-0013754

(51) Int. Cl. H01M 10/0567; H01M 10/0568; H01M 10/0569; H01M 10/0525; H01M 4/525; C07C 317/08; C07F 9/6574; C07F 5/04; C07C 309/66; C07C 309/65

(52) U.S. Cl. CPC H01M 10/0567; H01M 10/0568; H01M 10/0569; H01M 10/0525; H01M 4/525; H01M 2004/028; C07F 9/65742; C07F 5/04; C07C 309/66; C07C 309/65; C07C 317/08

(57) ABSTRACT

An electrolyte including: a lithium salt, a non-aqueous solvent, an alkyl sulfonate compound represented by Formula 2, and an unsaturated sulfone compound represented by Formula 3:

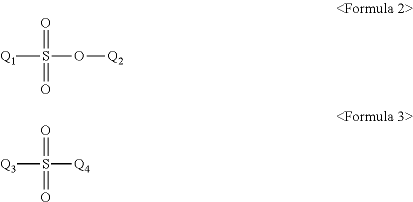

wherein, in Formula 2, Q₁ and Q₂ are each independently a substituted or unsubstituted C₁-C₂₀ alkyl group, and in Formula 3, Q₃ and Q₄ are each independently a group represented by -(L₁)-(R₁), a vinyl group, an allyl group, or a substituted or unsubstituted C₁-C₂₀ alkyl group, and at least one of Q₃ or Q₄ is a group represented by -(L₁)-(R₁), a vinyl group, or an allyl group, Li is a substituted or unsubstituted C₂-C₂₀ alkenylene group or a substituted or unsubstituted C₂-C₂₀ alkynylene group, and Ri is hydrogen or a substituted or unsubstituted C₂-C₂₀ alkyl group.

### CROSS-REFERENCE TO RELATED APPLICATION

This application claims priority to and the benefit of Korean Patent Application No. 10-2019-0013754, filed on Feb. 1, 2019, in the Korean Intellectual Property Office, and all the benefits accruing therefrom under 35 U.S.C. § 119, the content of which is incorporated herein by reference in its entirety.

### BACKGROUND

### 1. Field

The present disclosure relates to an electrolyte and a lithium battery including the electrolyte.

### 2. Description of the Related Art

A lithium battery is used as power source for a portable electronic device including a video camera, a mobile phone, a notebook computer, and the like. A lithium secondary battery, which is rechargeable, has a specific energy that is at least three times greater than that of a lead storage battery, a nickel-cadmium battery, a nickel-hydrogen battery, and a nickel-zinc battery, and is able to be charged at a high rate.

To manufacture a lithium secondary battery having a high energy density, a positive electrode active material exhibiting enhanced discharge capacity may be used. These positive electrode active materials have low electrochemical stability due to a side reaction between the positive electrode active material and an electrolyte, which occurs during charge and discharge of a lithium secondary battery, and results in deteriorated stability of the lithium secondary battery. Therefore, there is a need for a lithium secondary battery having enhanced stability and including a positive electrode active material with enhanced discharge capacity.

### SUMMARY

Additional aspects will be set forth in part in the description which follows and, in part, will be apparent from the description, or may be learned by practice of the presented embodiments.

According to an embodiment an electrolyte includes: a lithium salt; a non-aqueous solvent; an alkyl sulfonate compound represented by Formula 2; and an unsaturated sulfone compound represented by Formula 3

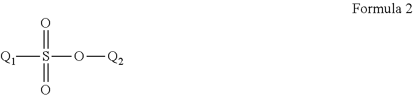

wherein, in Formula 2, Q₁ and Q₂ are each independently a substituted or unsubstituted C₂-C₂₀ alkyl group, and

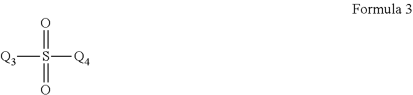

wherein, in Formula 3,

Q₃ and Q₄ are each independently a group represented by -(L₁)-(R₁), a vinyl group, an allyl group, or a substituted or unsubstituted C₁-C₂₀ alkyl group, and at least one of Q₃ or Q₄ is a group represented by -(L₁)-(R₁), a vinyl group, or an allyl group,

L₁ is a substituted or unsubstituted C₂-C₂₀ alkenylene group or a substituted or unsubstituted C₂-C₂₀ alkynylene group, and

R₁ is hydrogen or a substituted or unsubstituted C₂-C₂₀ alkyl group.

According to an aspect of an embodiment, a lithium battery includes:

a positive electrode;

a negative electrode; and

an electrolyte disposed between the positive electrode and the negative electrode,

wherein the positive electrode includes a positive electrode active material represented by Formula 1A, and

the electrolyte includes a lithium salt, a non-aqueous solvent, and an alkyl sulfonate compound represented by Formula 2

LiₓNi_{y}M_{1-y}O_{2-z}A_{z} Formula 1A

wherein, in Formula 1A, 0.9≤x≤1.2, 0.88≤y≤0.98, and 0≤z<0.2,

M is at least one of aluminum (Al), magnesium (Mg), manganese (Mn), cobalt (Co), iron (Fe), chromium (Cr), vanadium (V), titanium (Ti), copper (Cu), boron (B), calcium (Ca), zinc (Zn), zirconium (Zr), niobium (Nb), molybdenum (Mo), strontium (Sr), antimony (Sb), tungsten (W), or bismuth (Bi); and

A is an element having an oxidation number of −1, −2, or −3;

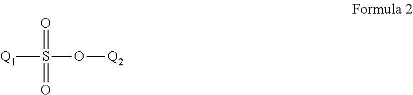

wherein, in Formula 2, Q₁ and Q₂ are each independently a substituted or unsubstituted C₁-C₂₀ alkyl group.

According to an aspect of another embodiment, a lithium battery includes:

a positive electrode;

a negative electrode; and

an electrolyte disposed between the positive electrode and the negative electrode, the electrolyte including a lithium salt, a non-aqueous solvent, an alkyl sulfonate compound, and an unsaturated sulfone compound,

wherein the positive electrode comprises a positive electrode active material represented by Formula 1B

LiₚNi_{q}M_{1-q}O₂₋ᵣAᵣ Formula 1B

wherein, in Formula 1B, 0.9≤p≤1.2, 0.7≤q≤0.98, and 0≤r<0.2 are satisfied;

M is at least one of aluminum (Al), magnesium (Mg), manganese (Mn), cobalt (Co), iron (Fe), chromium (Cr), vanadium (V), titanium (Ti), copper (Cu), boron (B), calcium (Ca), zinc (Zn), zirconium (Zr), niobium (Nb), molybdenum (Mo), strontium (Sr), antimony (Sb), tungsten (W), or bismuth (Bi); and

A is an element having an oxidation number of −1, −2, or −3.

Also disclosed is a method of manufacturing a lithium battery, the method including: providing a positive electrode and a negative electrode; and disposing the electrolyte between the positive electrode and the negative electrode.

### BRIEF DESCRIPTION OF THE DRAWINGS

These and/or other aspects will become apparent and more readily appreciated from the following description of the embodiments, taken in conjunction with the accompanying drawing in which:

FIG. 1 is a view of a lithium battery according to an example embodiment.

### DETAILED DESCRIPTION

The invention now will be described more fully hereinafter with reference to the accompanying drawings, in which various embodiments are shown. This invention may, however, be embodied in many different forms, and should not be construed as limited to the embodiments set forth herein. Rather, these embodiments are provided so that this disclosure will be thorough and complete, and will fully convey the scope of the invention to those skilled in the art. Like reference numerals refer to like elements throughout.

It will be understood that when an element is referred to as being “on” another element, it can be directly on the other element or intervening elements may be present therebetween. In contrast, when an element is referred to as being “directly on” another element, there are no intervening elements present.

The terminology used herein is for the purpose of describing particular embodiments only and is not intended to be limiting. As used herein, the singular forms “a,” “an,” “the,” and “at least one” do not denote a limitation of quantity, and are intended to include both the singular and plural, unless the context clearly indicates otherwise. For example, “an element” has the same meaning as “at least one element,” unless the context clearly indicates otherwise. “Or” means “and/or.” As used herein, the term “and/or” includes any and all combinations of one or more of the associated listed items. It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

“About” or “approximately” as used herein is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” can mean within one or more standard deviations, or within ±30%, 20%, 10% or 5% of the stated value.

Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

Exemplary embodiments are described herein with reference to cross section illustrations that are schematic illustrations of idealized embodiments. As such, variations from the shapes of the illustrations as a result, for example, of manufacturing techniques and/or tolerances, are to be expected. Thus, embodiments described herein should not be construed as limited to the particular shapes of regions as illustrated herein but are to include deviations in shapes that result, for example, from manufacturing. For example, a region illustrated or described as flat may, have rough and/or nonlinear features. Moreover, sharp angles that are illustrated may be rounded. Thus, the regions illustrated in the figures are schematic in nature and their shapes are not intended to illustrate the precise shape of a region and are not intended to limit the scope of the present claims.

Hereinafter, a lithium battery according to example embodiments, an electrolyte for a lithium battery, and a lithium battery employing the electrolyte will be described in more detail.

A lithium battery according to an embodiment includes a positive electrode, a negative electrode, and an electrolyte disposed between the positive electrode and the negative electrode, wherein the positive electrode includes a positive electrode active material represented by Formula 1A, and the electrolyte includes a lithium salt, a non-aqueous solvent, and an alkyl sulfonate compound represented by Formula 2,

LiₓNi_{y}M_{1-y}O_{2-z}A_{z} Formula 1A

wherein, in Formula 1A, 0.9≤x≤1.2, 0.88≤y≤0.98, and 0≤z<0.2 are satisfied,

M is at least one of aluminum (Al), magnesium (Mg), manganese (Mn), cobalt (Co), iron (Fe), chromium (Cr), vanadium (V), titanium (Ti), copper (Cu), boron (B), calcium (Ca), zinc (Zn), zirconium (Zr), niobium (Nb), molybdenum (Mo), strontium (Sr), antimony (Sb), tungsten (W), or bismuth (Bi); and

A is an element having an oxidation number of −1, −2, or −3;

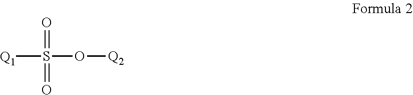

wherein, in Formula 2, Q₁ and Q₂ are each independently a substituted or unsubstituted C₂-C₂₀ alkyl group.

When the positive electrode active material includes a lithium metal composite oxide having a high Ni content, such as the positive electrode active material of Formula 1A, a high-output and high-capacity battery may be manufactured. However, Ni cations included in the lithium metal composite oxide may elute from a positive electrode into the electrolyte, or Li on a surface of the positive electrode reacts with oxygen or the like, thus deteriorating the positive electrode. Moreover, the Ni cations may react with a solid electrolyte interface (SEI) film on a negative electrode, thereby degrading the SEI film such that a negative electrode active material is partially exposed to the electrolyte, and consequently, a side reaction may occur, resulting in deteriorated capacity and lifespan characteristics of the lithium battery and an increase in the amount of gas generation, particularly at elevated temperatures, e.g., above 40° C. or more, or above 60° or more.

To address these issues, the lithium battery includes an electrolyte including the alkyl sulfonate compound of Formula 2, and thus a side reaction by Ni cations is minimized and consequently, gas generation is reduced, resulting in enhanced lifespan of the lithium battery.

In particular, the alkyl sulfonate compound is understood to have a high affinity for Ni cations, and maintains a high affinity for the Ni cations even when the battery is operated at a high voltage. As a result, the alkyl sulfonate compound suppresses side reactions between the Ni cations and the SEI thereby inhibiting the degradation of an SEI film.

In addition, the alkyl sulfonate compound may be adsorbed onto a surface of a positive electrode by interaction with Li cations, and thus may protect the surface of the positive electrode from oxidation. Thus, the alkyl sulfonate compound may reduce gas generation due to a side reaction at the surface of the positive electrode and lower battery resistance, thereby enhancing battery performance and lifespan.

In this regard, the amount of the alkyl sulfonate compound in the electrolyte may be 5 parts by weight or less with respect to 100 parts by weight of the electrolyte. That is, the amount of the alkyl sulfonate compound may be within a range that enables stabilization of Ni cations eluted into an electrolyte from a positive electrode active material and protection of the surface of the positive electrode by interaction with Li cations in the electrolyte. When the amount of the alkyl sulfonate compound exceeds 5 parts by weight, the alkyl sulfonate compound is decomposed, film resistance is increased, and production of CO₂ gas has an adverse effect, and accordingly, battery capacity, storage stability, and cycle characteristics may deteriorate.

According to an embodiment, the amount of the alkyl sulfonate compound may be in a range of about 0.005 parts by weight to about 5 parts by weight with respect to 100 parts by weight of the electrolyte. For example, the amount of the alkyl sulfonate compound may be in a range of about 0.001 parts by weight to about 3 parts by weight, about 0.01 parts by weight to about 2 parts by weight, or about 0.1 parts by weight to about 1.5 parts by weight, with respect to 100 parts by weight of the electrolyte.

When the amount of the alkyl sulfonate compound is less than 0.005 parts by weight with respect to 100 parts by weight of the electrolyte, the amount is excessively small, such that it may be difficult to sufficiently obtain an effect of protecting a positive electrode.

According to an embodiment, in Formula 2, Q₁ and Q₂ may be each independently a methyl group, an ethyl group, a propyl group, an isopropyl group, a butyl group, a sec-butyl group, a tert-butyl group, or an isobutyl group; or

a methyl group, an ethyl group, a propyl group, an isopropyl group, a butyl group, a sec-butyl group, a tert-butyl group, or an isobutyl group, each of which is substituted with at least one of deuterium, —F, —Cl, —Br, —I, a cyano group (—CN), a nitro group (—NO₂), a hydroxyl group (—OH), a methyl group (—CH₃), an ethyl group (—CH₂CH₃), or a propyl group (—(CH₂)₂CH₃).

For example, the alkyl sulfonate compound may be at least one of compounds 1 or 2

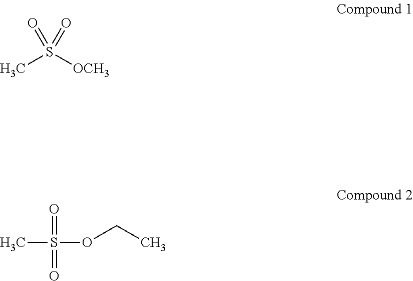

The alkyl sulfonate compound of Formula 2 may reduce gas generation of a lithium battery resulting from a high oxidation voltage, may protect the surface of the positive electrode from oxygen by being adsorbed onto the surface of the positive electrode during charge, and suppresses the degradation of an SEI film by stabilizing Ni cations. Consequently, gas generation from a side reaction at the surface of the positive electrode is reduced and battery resistance is lowered, and a lithium battery may exhibit enhanced performance and lifespan characteristics.

The electrolyte includes a lithium salt. The lithium salt may act as a supply source of lithium ions in a battery by being dissolved in an organic solvent, and for example, may serve to promote the transfer of lithium ions between a positive electrode and a negative electrode.

An anion of the lithium salt included in the electrolyte may be at least one of PF₆⁻, BF₄⁻, SbF₆⁻, AsF₆⁻, C₄F₉SO₃⁻, ClO₄⁻, AlO₂−, AlCl₄⁻, CₓF₂ₓ₊₁SO₃⁻ (wherein, x is a natural number, for example, 1 to 10), (CₓF₂ₓ₊₁SO₂)(C_{y}F_{2y+1}SO₂)N⁻ (wherein, x and y are natural numbers, for example, independently 1 to 10), or a halide.

In the electrolyte, the lithium salt may include at least one of LiPF₆, LiBF₄, LiCF₃SO₃, Li(CF₃SO₂)₂N, LiC₂F₅SO₃, Li(FSO₂)₂N, LiC₄F₉SO₃, or LiN(SO₂CF₂CF₃)₂, a compound represented by Formula 22, a compound represented by Formula 23, a compound represented by Formula 24, or a compound represented by Formula 25 below:

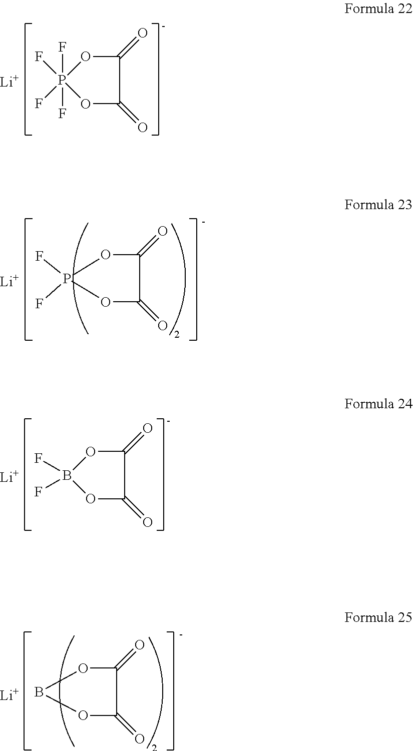

The concentration of the lithium salt may be in a range of about 0.01 moles per liter (M) to about 5 M, about 0.05 M to about 5 M, about 0.1 M to about 5 M, or about 0.1 M to about 2 M, but is not limited thereto, and an appropriate concentration of the lithium salt may be determined by a person of skill in the art without undue experimentation.

The amount of the lithium salt in a solvent-free electrolyte may be in a range of about 0.001 parts by weight to about 30 parts by weight, with respect to 100 parts by weight of the solvent-free electrolyte, but is not limited thereto, and the lithium salt may be used in any suitable amount that enables the electrolyte to effectively transfer lithium ions and/or electrons during charge and discharge processes.

The amount of the lithium salt in a solvent-containing electrolyte may be in a range of about 100 millimoles per liter (mM) to about 10 M. For example, the amount of the lithium salt may be in a range of about 100 mM to about 2 M. For example, the amount of the lithium salt may be in a range of about 500 mM to about 2 M. However, the amount is not limited thereto and the lithium salt may be used within any amount that enables the electrolyte to effectively transfer lithium ions and/or electrons during charge and discharge processes.

According to an embodiment, the amount of the lithium salt in the electrolyte may be in a range of about 1.1 M to about 2.5 M. For example, the concentration of the lithium salt may be in a range of about 1.15 M to about 2.2 M, or about 1.3 M to about 2 M.

The non-aqueous solvent may include at least one of a carbonate-based solvent, an ester-based solvent, an ether-based solvent, a ketone-based solvent, or an aprotic solvent.

The carbonate-based solvent may be a chain (linear or branched) carbonate solvent or a cyclic carbonate solvent. Non-limiting examples of the carbonate-based (e.g. carbonate-containing) solvent include dimethyl carbonate (DMC), diethyl carbonate (DEC), ethyl methyl carbonate (EMC), dipropyl carbonate (DPC), methyl propyl carbonate (MPC), ethyl propyl carbonate (EPC), methyl ethyl carbonate (MEC), ethylene carbonate (EC), propylene carbonate (PC), butylene carbonate (BC), and tetraethylene glycol dimethyl ether (TEGDME). Non-limiting examples of the ester-based (e.g. ester-containing) solvent include methyl acetate, ethyl acetate, n-propyl acetate, dimethyl acetate, methyl propionate (MP), ethyl propionate, γ-butyrolactone, decanolide, valerolactone, mevalonolactone, and caprolactone. Non-limiting examples of the ether-based (e.g. ether-containing) solvent include dibutyl ether, tetraglyme, diglyme, dimethoxyethane, 2-methyl tetrahydrofuran, and tetrahydrofuran. An example of the ketone-based (e.g. ketone-containing) solvent may be cyclohexanone or the like. As a nitrile-based (e.g. nitrile-containing) solvent, acetonitrile (AN), succinonitrile (SN), adiponitrile, or the like may be used.

The aprotic solvents may be used alone or at least one of these solvents may be used in combination comprising at least one of the aprotic solvents. In a case in which at least one of these solvents are combined, a mixing ratio may be selected by a person of skill in the art without undue experimentation.

Additional solvents may also be used, such as, dimethylsulfoxide, dimethylformamide, dimethylacetamide, tetrahydrofuran, and the present disclosure is not limited thereto, and any suitable organic solvent suitable for use in a lithium battery may be used.

For example, the non-aqueous solvent may include about 50 volume percent (vol %) to about 95 vol % of a chain carbonate and about 5 vol % to about 50 vol % of a cyclic carbonate; about 55 vol % to about 95 vol % of a chain carbonate and about 5 vol % to about 45 vol % of a cyclic carbonate; about 60 vol % to about 95 vol % of a linear carbonate and about 5 vol % to about 40 vol % of a cyclic carbonate; about 65 vol % to about 95 vol % of a linear carbonate and about 5 vol % to about 35 vol % of a cyclic carbonate; or about 70 vol % to about 95 vol % of a chain carbonate and about 5 vol % to about 30 vol % of a cyclic carbonate. For example, the non-aqueous solvent may be a mixture including three or more non-aqueous solvents.

In an embodiment, the non-aqueous solvent may further include at least one of fluoro-ethylene carbonate (FEC), vinylene carbonate (VC), vinyl ethylene carbonate (VEC), a phosphorus (P)-containing compound, or a sulfur (S)-containing compound. An amount of the FEC, VC, VEC, the P-containing compound, or the S-containing compound may be about 0.1 vol % to about 10 vol %, or about 0.5 vol % to about 7 vol %, or about 1 vol % to about 7 vol %, or about 2 vol % to about 7 vol %, with respect to a total volume of the non-aqueous solvent.

For example, the non-aqueous solvent may include FEC. For example, the lithium battery may include FEC in an amount of about 0.1 vol % to about 10 vol %, with respect to a total volume of the non-aqueous solvent. For example, the lithium battery may include the FEC in an amount of about 0.5 vol % to about 7 vol % with respect to the total volume of the non-aqueous solvent. For example, the lithium battery may include the FEC in an amount of about 1 vol % to about 7 vol % with respect to the total volume of the non-aqueous solvent. For example, the lithium battery may include the FEC in an amount of about 2 vol % to about 7 vol % with respect to the total volume of the non-aqueous solvent. When the amount of the FEC in the non-aqueous solvent is within the above ranges, an effective SEI film that does not hinder a diffusion rate of lithium ions may be rapidly formed.

According to an embodiment, the non-aqueous solvent may include at least one of dimethyl carbonate (DMC), diethyl carbonate (DEC), ethyl methyl carbonate (EMC), dipropyl carbonate (DPC), methyl propyl carbonate (MPC), ethyl propyl carbonate (EPC), methyl ethyl carbonate (MEC), ethylene carbonate (EC), propylene carbonate (PC), butylene carbonate (BC), methyl propionate (MP), ethyl propionate, propyl propionate (PP), or tetraethylene glycol dimethyl ether (TEGDME).

The electrolyte may include at least one of a carbonate-based solvent containing a carbon-carbon single bond, a carbon-carbon double bond, or a carbon-carbon triple bond, or a carboxylic acid anhydride containing a carbon-carbon single bond, a carbon-carbon double bond, or a carbon-carbon triple bond, or a combination thereof. The carboxylic acid anhydride may be a linear carboxylic acid anhydride or a cyclic carboxylic acid anhydride. A combination comprising at least one of the foregoing solvents may be used.

According to an embodiment, the electrolyte may include at least one of a cyclic anhydride compound, a cyclic acid anhydride compound, a P-containing compound, or an S-containing compound.

According to an embodiment, the electrolyte includes at least one of a cyclic anhydride compound or a cyclic acid anhydride compound, and the alkyl sulfonate compound may have a reduction potential which is greater than a reduction potential of the cyclic anhydride compound and/or a reduction potential of the cyclic acid anhydride compound.

In addition, according to an embodiment, an amount of the cyclic anhydride compound, the cyclic acid anhydride compound, or the combination thereof may be in a range of about 0.1 parts by weight to about 2 parts by weight, or about 0.5 parts by weight to about 2 parts by weight, or about 0.5 parts by weight to about 1.5 parts by weight, with respect to 100 parts by weight of the electrolyte.

The cyclic anhydride compound may be, for example, at least one fluoro-ethylene carbonate (FEC), vinylene carbonate (VC), and vinyl ethylene carbonate (VEC).

The cyclic acid anhydride compound may include, for example, at least one of maleic anhydride or succinic anhydride.

The P-containing compound may include, for example, at least one of a phosphine compound, a phosphate compound, or a phosphite compound.

In particular, the phosphine compound may be at least one of triphenylphosphine, tris(4-fluorophenyl)phosphine, tris(2,4-difluorophenyl)phosphine, or tris(perfluorophenyl)phosphine, but is not limited thereto. The phosphate compound may be particularly at least one of triphenyl phosphate (TPPa) or trimethyl phosphate (TMPa), but is not limited thereto. The phosphite compound may be particularly at least one of triethylphosphite (TEPi), trimethylphosphite, tripropylphosphite, tributylphosphite, tris(trimethylsillyl)phosphite, or triphenylphosphite, but is not limited thereto.

The S-containing compound may include, for example, at least one of a sulfone compound, a sulfonate compound, a sultone compound, or a disulfonate compound.

In particular, the sulfone compound may be at least one of ethylmethyl sulfone, divinyl sulfone, or tetramethylene sulfone, but is not limited thereto. The sulfonate compound may be at least one of methyl methane sulfonate, ethyl methane sulfonate, or diallyl sulfonate, but is not limited thereto. The disulfonate compound may be at least one of methylene methane disulfonate (MMDS) or bisulfan, but is not limited thereto. The sultone compound may be fluoropropane sultone (FPS), but is not limited thereto.

According to an embodiment, the lithium battery may include the electrolyte in an amount of about 1 gram per ampere-hour (g/Ah) to about 3 g/Ah.

The positive electrode includes the positive electrode active material of Formula 1A.

For example, in Formula 1A, A may be any one of a halogen, sulfur (S), and nitrogen (N).

In Formula 1A, y denotes the content of nickel (Ni) in the positive electrode active material. According to an embodiment, in Formula 1A, y may satisfy the following condition: 0.88≤y≤0.98.

In addition, according to an embodiment, in Formula 1A, M may be at least one of Co, Ni, or Mn.

For example, when the positive electrode includes the positive electrode active material of Formula 1A, the positive electrode may include at least one of Li_{1.02}Ni_{0.88}Co_{0.08}Mn_{0.04}O₂, Li_{1.02}N i_{0.88}Co_{1.10}Mn_{0.02}O₂, Li_{1.02}Ni_{0.91}Co_{0.06}Mn_{0.03}O₂, LiNi_{0.94}Co_{0.04}Mn_{0.02}O₂, Li_{1.02}Ni_{0.88}Co_{0.08}Al_{0.04}O₂, Li_{1.02}Ni_{0.88}Co_{0.10}Al_{0.02}O₂, Li_{1.02}Ni_{0.91}Co_{0.06}Al_{0.03}O₂, or LiNi_{0.94}Co_{0.04}Al_{0.02}O₂.

For example, the positive electrode active material may be represented by Formula 10 or 20:

LiₓNi_{y}Co_{1-y′-z′}Al_{z′}O₂ Formula 10

LiₓNi_{y′}Co_{1-y′-z′}Mn_{z′}O₂ Formula 20

In Formula 10 and Formula 20, 0.9≤x′≤1.2, 0.88≤y′≤0.98, 0<z′<0.1, and 0<1−y′−z′<0.2.

As discussed above, in the case of a lithium metal oxide having a high Ni content, a high-capacity battery may be realized, however, as the content of Ni cations in the battery increases, there is a deterioration in the lifespan characteristics of the battery.

In addition, as described below, in a lithium battery including a negative electrode active material including a metal alloyable with lithium or a carbon-based negative electrode active material, at gas is generated, e.g., due to catalytic action at a high temperature, and accordingly, the lithium battery exhibits deteriorated lifespan characteristics.

An amount of each of fluoro-ethylene carbonate, vinylene carbonate, vinyl ethylene carbonate, maleic anhydride, succinic anhydride, the phosphorus-containing compound, or the sulfur-containing compound is in a range of about 0.1 parts by weight to about 2 parts by weight, with respect to 100 parts by weight of the electrolyte. As described above, when the amount of FEC, VC, VEC, MA, SA, the P-containing compound, or the S-containing compound is within the above described ranges, a passivation film (e.g., an SEI film) including a chemical reaction product of the above material may be formed on a portion or all of a surface of the negative electrode.

In addition, the positive electrode may further include, in addition to the above-listed positive electrode active materials, at least one of lithium cobalt oxide, lithium nickel cobalt manganese oxide, lithium nickel cobalt aluminum oxide, lithium iron phosphate, or lithium manganese oxide, but is not particularly limited thereto, and the positive electrode may further include any suitable positive electrode active material art.

The negative electrode includes a negative electrode active material, and the negative electrode active material may include at least one of a silicon-based compound, a carbon-based compound, a composite of a silicon-based compound and a carbon-based compound, or a silicon oxide (SiOₓ₁, where 0<x1<2). In another embodiment, for example, the negative electrode may include a negative electrode active material including a metal alloyable with lithium, a silicon-based negative electrode active material, and/or a carbon-based negative electrode active material.

For example, the silicon-based compound may include silicon particles having an average diameter of 200 nanometers (nm) or less.

For example, the carbon-based compound may include graphite.

For example, the composite of a silicon-based compound and a carbon-based compound may be a composite having a structure in which silicon nanoparticles are arranged on the carbon-based compound, a composite in which silicon particles are included on a surface of and inside the carbon-based compound, or a composite in which silicon particles are coated with the carbon-based compound and thereby included in the carbon-based compound. The composite of a silicon-based compound and a carbon-based compound may be an active material obtained by dispersing silicon nanoparticles having an average diameter of about 200 nm or less on carbon-based compound particles, and then coating the resulting structure with carbon, an active material in which silicon particles are present on and inside graphite. Secondary particles of the composite of a silicon-based compound and a carbon-based compound may have an average diameter of about 5 micrometers (μm) to about 20 μm, and the silicon nanoparticles may have an average diameter of 200 nm or less, 150 nm or less, 100 nm or less, 50 nm or less, 20 nm or less, or 10 nm or less. For example, the average diameter of the silicon nanoparticles may be in a range of about 1 nm to about 200 nm or about 10 nm to about 150 nm, or about 100 nm to about 150 nm.

For example, the composite of a silicon-based compound and a carbon-based compound may have a capacity of about 300 milliampere-hours per gram (mAh/g) to about 700 mAh/g. For example, the capacity of the composite of a silicon-based compound and a carbon-based compound may be in a range of about 350 mAh/g to about 650 mAh/g, or about 400 mAh/g to about 600 mAh/g.

The lithium battery may have a capacity retention rate of 80% or greater, for example, 82% or greater, after 200 cycles of charge and discharge at 25° C. For example, when the negative electrode of the lithium battery includes a silicon compound or a silicon oxide, the capacity retention rate of the lithium battery after 200 cycles of charge and discharge at 25° C. may be 85% or greater.

The lithium battery may have a direct current internal resistance (DCIR) increase rate of 150% or less after 200 cycles of charge and discharge at 25° C. For example, when the negative electrode of the lithium battery includes a silicon compound or a silicon oxide, the DCIR increase rate of the lithium battery after 200 cycles of charge and discharge at 25° C. may be 135% or less, for example, 125% or less.

The lithium battery may have a DCIR increase rate of 100% or less after being stored at 60° C. for 10 days. That is, a lithium battery including the alkyl sulfonate compound in the electrolyte and having a Ni content of 88 wt % or higher in the positive electrode active material may exhibit a substantially reduced DCIR when stored at a high temperature.

The lithium battery may have a cell energy density per unit volume of about 500 Watt-hours per liter (Wh/L) or greater, for example 600 Wh/L, or about 750 Wh/L. Since the lithium battery provides a high energy density, i.e., 500 Wh/L or greater, high output may be obtained.

According to another embodiment, there is provided an electrolyte including: a lithium salt; a non-aqueous solvent; an alkyl sulfonate compound represented by Formula 2; and an unsaturated sulfone compound represented by Formula 3

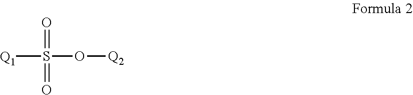

wherein, in Formula 2, Q₁ and Q₂ are each independently a substituted or unsubstituted C₁-C₂₀ alkyl group,

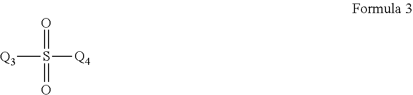

wherein, in Formula 3, Q₃ and Q₄ are each independently a group represented by -(L₁)-(R₁), a vinyl group, an allyl group, or a substituted or unsubstituted C₁-C₂₀ alkyl group, and at least one of Q₃ and Q₄ is a group represented by -(L₁)-(R₁), a vinyl group, or an allyl group,

L₁ is a substituted or unsubstituted C₂-C₂₀ alkenylene group or a substituted or unsubstituted C₂-C₂₀ alkynylene group, and

R₁ is hydrogen or a substituted or unsubstituted C₂-C₂₀ alkyl group.

When a lithium metal composite oxide having a high Ni content, such as the positive electrode active material of Formula 1B is used, a high-output and high-capacity battery may be manufactured, but Ni cations included in the lithium metal composite oxide are eluted from a positive electrode into an electrolyte, or Li on a surface of the positive electrode reacts with oxygen, thus deteriorating the positive electrode. Further, the Ni cations react with an SEI film on a negative electrode, thereby degrading the SEI film, and accordingly, the negative electrode active material is partially exposed to the electrolyte thus causing a side reaction, resulting in deteriorated capacity and lifespan characteristics and an increase in the amount of gas generated.

To address these issues, the lithium battery includes an electrolyte including the alkyl sulfonate compound of Formula 2 and the unsaturated sulfone compound of Formula 3, and thus a side reaction between the Ni cations and the SEI film is minimized and consequently, gas generation is reduced, resulting in enhanced battery lifespan.

In particular, the alkyl sulfonate compound has a high affinity for the Ni cations, and thus has an effect of suppressing a side reaction of the Ni cations, and in particular, maintains a high affinity for the Ni cations even when a battery is operated at a high voltage, thus having an effect of inhibiting the degradation of an SEI film.

In addition, the alkyl sulfonate compound may be adsorbed onto a surface of a positive electrode by interaction with Li cations, and thus may protect the surface of the positive electrode from oxygen. Thus, the alkyl sulfonate compound may reduce gas generation due to a side reaction at the surface of the positive electrode and lower battery resistance, thereby enhancing battery performance and lifespan.

In addition, the unsaturated sulfone compound may be reduced and decomposed at a metallic negative electrode before the solvent, thereby forming a more stable SEI film at the surface of the negative electrode. The SEI film formed on the surface of the negative electrode may reduce gas generation caused by a side reaction, thereby enhancing electrochemical characteristics of a battery.

Thus, the electrolyte including the alkyl sulfonate compound and the unsaturated sulfone compound protects a positive electrode and enhances the stability of an SEI film, and thus gas generation of a lithium battery may be reduced and battery resistance may be lowered, resulting in enhanced battery performance and lifespan.

According to an embodiment, the amount of the alkyl sulfonate compound may be in a range of about 0.001 parts by weight to about 3 parts by weight, with respect to 100 parts by weight of the electrolyte. For example, the amount of the alkyl sulfonate compound may be in a range of about 0.01 parts by weight to about 1.5 parts by weight, or about 0.1 parts by weight to about 0.5 parts by weight, with respect to 100 parts by weight of the electrolyte.

When the amount of the alkyl sulfonate compound is less than 0.001 parts by weight with respect to 100 parts by weight of the electrolyte, the amount is excessively small, such that it may be difficult to effectively protect a positive electrode.

According to an embodiment, the amount of the unsaturated sulfone compound may be in a range of about 0.001 parts by weight to about 3 parts by weight with respect to 100 parts by weight of the electrolyte. For example, the amount of the unsaturated sulfone compound may be in a range of about 0.01 parts by weight to about 1.5 parts by weight, or about 0.1 parts by weight to about 0.5 parts by weight, with respect to 100 parts by weight of the electrolyte.

When the amount of the unsaturated sulfone compound is less than 0.001 parts by weight with respect to 100 parts by weight of the electrolyte, the amount is excessively small, such that the SEI film may not be formed and it may be difficult to effectively reduce resistance of the battery.

According to an embodiment, the sum of the amount of the alkyl sulfonate compound and the amount of the unsaturated sulfone compound may be in a range of about 0.001 parts by weight to about 5 parts by weight, with respect to 100 parts by weight of the electrolyte. For example, the sum of the amount of the alkyl sulfonate compound and the amount of the unsaturated sulfone compound may be in a range of about 0.005 parts by weight to about 3 parts by weight, or about 0.01 parts by weight to about 2 parts by weight, or about 0.1 parts by weight to about 1.5 parts by weight, with respect to 100 parts by weight of the electrolyte.

When the sum of the amount of the alkyl sulfonate compound and the amount of the unsaturated sulfone compound is less than 0.001 parts by weight with respect to 100 parts by weight of the electrolyte, the amount is excessively small, the SEI film may not be formed and it may be difficult to effectively protect a positive electrode and reduce resistance of the battery.

According to an embodiment, in Formula 2, Q₁ and Q₂ may be each independently a methyl group, an ethyl group, a propyl group, an isopropyl group, a butyl group, a sec-butyl group, a tert-butyl group, or an isobutyl group; or a methyl group, an ethyl group, a propyl group, an isopropyl group, a butyl group, a sec-butyl group, a tert-butyl group, or an isobutyl group, each of which is substituted with at least one of deuterium, —F, —Cl, —Br, —I, a cyano group (—CN), a nitro group (—NO₂), a hydroxyl group (—OH), a methyl group (—CH₃), an ethyl group (—CH₂CH₃), or a propyl group (—(CH₂)₂CH₃).

According to an embodiment, in Formula 3, Q₃ and Q₄ may be each independently a vinyl group, an allyl group, a methyl group, an ethyl group, a propyl group, an isopropyl group, a butyl group, a sec-butyl group, a tert-butyl group, or an isobutyl group; or a methyl group, an ethyl group, a propyl group, an isopropyl group, a butyl group, a sec-butyl group, a tert-butyl group, or an isobutyl group, each of which is substituted with at least one of deuterium, —F, —Cl, —Br, —I, a cyano group, a nitro group, a hydroxyl group, a methyl group, an ethyl group, or a propyl group, and at least one of Q₃ and Q₄ may be a vinyl group or an allyl group.

For example, the alkyl sulfonate compound may be at least one of Compound 1 or Compound 2

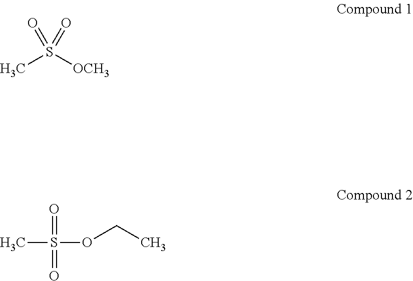

For example, the unsaturated sulfone compound may be Compound 3.

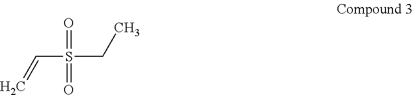

The alkyl sulfonate compound of Formula 2 may reduce gas generation of a lithium battery due to a high oxidation voltage thereof, protect the surface of the positive electrode from oxygen by being adsorbed onto the surface of the positive electrode during charge, and suppress the degradation of the SEI film by stabilizing Ni cations. Accordingly, the presence of a compound of Formula 2 may reduce gas generation caused by a side reaction at the surface of the positive electrode and lowering battery resistance. Accordingly, the lithium battery may exhibit enhanced performance and lifespan characteristics.

In addition, the unsaturated sulfone compound of Formula 3 may form, on the surface of the negative electrode, a stable SEI film capable of capturing Ni cations eluted from the positive electrode to thereby protect the negative electrode. Thus, when the electrolyte including the alkyl sulfonate compound and the unsaturated sulfone compound is applied, a lithium battery may have a reduced initial resistance and a resistance of the lithium battery is relatively lower after storage at a high temperature. Accordingly, the lithium battery may exhibit enhanced lifespan characteristics.

According to an embodiment, the electrolyte does not include an aromatic hydrocarbon or an aromatic hydrocarbon compound substituted with a halogen. As used herein, an aromatic hydrocarbon refers to a hydrocarbon compound comprising at least one unsaturated cyclic group having delocalized pi electrons.

The electrolyte includes a lithium salt, such as those previously described herein.

The electrolyte includes a non-aqueous solvent as previously described herein.

According to an embodiment, as described above, the electrolyte includes a cyclic anhydride compound, a cyclic acid anhydride compound, or a mixture thereof, and the unsaturated sulfone compound may have a reduction potential which is greater than the reduction potential of the cyclic anhydride compound or the reduction potential of the cyclic acid anhydride compound.

According to an embodiment, there is provided a lithium battery including a positive electrode, a negative electrode, and an electrolyte disposed between the positive electrode and the negative electrode,

wherein the electrolyte includes a lithium salt, a non-aqueous solvent, an alkyl sulfonate compound, and an unsaturated sulfone compound, and

the positive electrode includes a positive electrode active material represented by Formula 1B

LiₚNi_{q}M_{1-q}O₂₋ᵣAᵣ Formula 1B

wherein, in Formula 1B, 0.9≤p≤1.2, 0.7≤q≤0.98, and 0≤r<0.2 are satisfied;

M is at least one of Al, Mg, Mn, Co, Fe, Cr, V, Ti, Cu, B, Ca, Zn, Zr, Nb, Mo, Sr, Sb, W, or Bi; and

A is an element having an oxidation number of −1, −2, or −3.

According to an embodiment, the amount of the electrolyte in the lithium battery may be in a range of about 1 gram per ampere-hour (g/Ah) to about 3 g/Ah.

The positive electrode includes the positive electrode active material of Formula 1B.

For example, in Formula 1B, A may be at least one of a halogen, S, or N.

In Formula 1B, q denotes the content of Ni in the positive electrode active material. According to an embodiment, in Formula 1B, q may satisfy the following condition: 0.7≤q≤0.98.

LiₚNi_{q}M_{1-q}O₂₋ᵣAᵣ Formula 1B

wherein, in Formula 1B, 0.9≤p≤1.2, 0.7≤q≤0.98, and 0≤r<0.2;

M is at least one of I, Mg, Mn, Co, Fe, Cr, V, Ti, Cu, B, Ca, Zn, Zr, Nb, Mo, Sr, Sb, W, or Bi; and

A is an element having an oxidation number of −1, −2, or −3.

According to an embodiment, in Formula 1B, q may satisfy the following condition: 0.8≤q≤0.98.

For example, the positive electrode may include at least one of Li_{1.02}Ni_{0.80}Co_{0.15}Mn_{0.05}O₂, Li_{1.02}Ni_{0.85}Co_{0.1}Mn_{0.05}O₂, Li_{1.02}Ni_{0.88}Co_{0.08}Mn_{0.04}O₂, Li_{1.02}Ni_{0.88}Co_{0.10}Mn_{0.02}O₂, Li_{1.02}Ni_{0.91}Co_{0.06}Mn_{0.03}O₂, LiNi_{0.94}Co_{0.04}Mn_{0.02}O₂, Li_{1.02}Ni_{0.80}Co_{0.15}Al_{0.05}O₂, Li_{1.02}Ni_{0.85}Co_{0.1}Al_{0.05}O₂, Li_{1.02}Ni_{0.88}Co_{0.08}Al_{0.04}O₂, Li_{1.02}Ni_{0.88}Co_{0.10}Al_{0.02}O₂, Li_{1.02}Ni_{0.91}Co_{0.06}Al_{0.03}O₂, or LiNi_{0.94}Co_{0.04}Al_{0.02}O₂.

According to an embodiment, the positive electrode active material of Formula 1B may be represented by at least one of Formula 30 or Formula 40

Li_{p′}Ni_{q′}Co_{1-q′-r′}Al_{r′}O₂ Formula 30

Li_{p′}Ni_{q′}Co_{1-q′-r′}Mn_{r′}O₂ Formula 40

wherein, in Formula 30 and Formula 40, 0.9≤p′≤1.2, 0.88≤q′≤0.98, 0<r′<0.1, 0<1−q′−r′<0.2.

For example, the positive electrode may include at least one of Li_{1.02}Ni_{0.88}Co_{0.08}Mn_{0.04}O₂, Li_{1.02}N i_{0.88}Co_{1.10}Mn_{0.02}O₂, Li_{1.02}Ni_{0.91}Co_{0.06}Mn_{0.03}O₂, LiNi_{0.94}Co_{0.04}Mn_{0.02}O₂, Li_{1.02}Ni_{0.88}Co_{0.08}Al_{0.04}O₂, Li_{1.02}Ni_{0.88}Co_{0.10}Al_{0.02}O₂, Li_{1.02}Ni_{0.91}Co_{0.06}Al_{0.03}O₂, or LiNi_{0.94}Co_{0.04}Al_{0.02}O₂.

As described above, when a lithium metal oxide having a high Ni content is used, a high-capacity battery may be manufactured, but the battery may exhibit poor lifespan characteristics as the amount of Ni cations increases.

In addition, as previously described, gas is generated in a lithium battery including a negative electrode active material including a metal alloyable with lithium or a carbon-based negative electrode active material, due to catalytic action at a high temperature, and accordingly, the lithium battery exhibits deteriorated lifespan characteristics.

As described previously, when the amount of FEC, VC, VEC, MA, SA, the P-containing compound, or the S-containing compound is within the above range, a passivation film (an SEI film) including a chemical reaction product of this material may be formed on a portion of the surface or on all of the surface of the negative electrode.

In this regard, the unsaturated sulfone compound may form a rigid SEI film capable of capturing Ni cations eluted from the positive electrode. In addition, since the alkyl sulfonate compound and the unsaturated sulfone compound are included in the electrolyte, Ni cations are stabilized, and thus gas generation when a battery is stored at a high temperature is minimized. Moreover, the battery may exhibit an increase in resistance due to the degradation of an SEI film. Accordingly, a battery may exhibit enhanced safety, lifespan, and performance.

In addition, the positive electrode may further include, in addition to the above-listed positive electrode active materials, at least one of lithium cobalt oxide, lithium nickel cobalt manganese oxide, lithium nickel cobalt aluminum oxide, lithium iron phosphate, or lithium manganese oxide, but is not limited thereto, and the positive electrode may further include any suitable positive electrode active material.

The negative electrode includes a negative electrode active material, and the negative electrode active material may include at least one of a silicon-based compound, a carbon-based compound, a composite of a silicon-based compound and a carbon-based compound, or a silicon oxide (SiOₓ₁, where 0<x1<2). In another embodiment, for example, the negative electrode may include a negative electrode active material including a metal alloyable with lithium, a silicon-based negative electrode active material, and/or a carbon-based negative electrode active material.

For example, the silicon-based compound may include silicon particles having an average diameter of about 10 nm to about 200 nm.

For example, the carbon-based compound may include graphite.

For example, the composite of a silicon-based compound and a carbon-based compound may be a composite having a structure in which silicon nanoparticles are disposed on a surface of the carbon-based compound, a composite in which silicon particles are disposed on a surface of the carbon-based compound and are coated with the carbon-based compound, or a composite in which silicon particles are coated with the carbon-based compound and thereby included in the carbon-based compound. The composite of a silicon-based compound and a carbon-based compound may be an active material obtained by dispersing silicon nanoparticles having an average diameter of about 200 nm or less on carbon-based compound particles, and then coating the resulting structure with carbon, to provide an active material in which silicon particles are disposed on the carbon-based compound and coated with carbon (e.g., graphite, or the like). Secondary particles of the composite of a silicon-based compound and a carbon-based compound may have an average diameter of about 5 micrometers (μm) to about 20 μm, and silicon nanoparticles may have an average diameter of 200 nm or less, 150 nm or less, 100 nm or less, 50 nm or less, 20 nm or less, or 10 nm or less. For example, the average diameter of the silicon nanoparticles may be in a range of about 1 nm to about 200 nm, or about 10 nm to about 150 nm, or about 100 nm to about 150 nm.

For example, the composite of a silicon-based compound and a carbon-based compound may have a capacity of about 300 mAh/g to about 700 mAh/g. For example, the capacity of the composite of a silicon-based compound and a carbon-based compound may be in a range of about 400 mAh/g to about 600 mAh/g.

The lithium battery may have a capacity retention rate of 80% or greater, for example, 85% or greater, after 100 cycles of charge and discharge at 25° C. For example, when the negative electrode of the lithium battery includes a silicon compound or a silicon oxide, the capacity retention rate of the lithium battery after 100 cycles of charge and discharge at 25° C. may be 90% or greater.

The lithium battery may have a DCIR increase rate of 130% or less after 300 cycles of charge and discharge at 25° C. For example, when the negative electrode of the lithium battery includes a silicon compound or a silicon oxide, the DCIR increase rate of the lithium battery after 200 cycles of charge and discharge at 25° C. may be 125% or less, for example, 120% or less.

The lithium battery may have a DCIR increase rate of 130% or less after being stored at 60° C. for 30 days. For example, the DCIR increase rate of the lithium battery after being stored at 60° C. for 10 days may be 110% or less.

The lithium battery may have a cell energy density per unit volume of 500 Wh/L or more. Since the lithium battery provides a high energy density, i.e., 500 Wh/L or higher, high output may be obtained.

The type of the lithium battery is not particularly limited, and the lithium battery includes a lithium-ion battery, a lithium-ion polymer battery, a lithium sulfur battery, and the like.

A lithium battery according to an embodiment may be manufactured using the following method.

First, a positive electrode is prepared.

For example, a positive electrode active material, a conductive material, a binder, and a solvent are mixed to prepare a positive electrode active material composition. The positive electrode active material composition may be directly coated onto a positive electrode current collector to thereby complete the manufacture of a positive electrode. In another embodiment, the positive electrode active material composition may be cast onto a separate support and a film separated from the support may be laminated on a metal current collector to thereby complete the manufacture of a positive electrode. The positive electrode is not limited to the above-described form, and may have a form other than the above-described form.

The positive electrode active material may be used in combination with another lithium-containing metal oxide, in addition to the positive electrode active material of Formula 1A or the positive electrode active material of Formula 1B. The lithium-containing metal oxide may be, for example, a composite oxide of lithium and at least one of Co, Mn, or Ni.

For example, the positive electrode active material may further include a lithium-containing metal oxide represented by any one of the following formulae: LiₐA_{1-b}B′_{b}D₂, where 0.90≤a≤1.8 and 0≤b≤0.5; LiₐE_{1-b}B′_{b}O_{2-c}D_{c}, where 0.90≤a≤1.8, 0≤b≤0.5, and 0≤c≤0.05; LiE_{2-b}B′_{b}O_{4-c}D_{c}, where 0≤b≤0.5 and 0≤c≤0.05; LiₐNi_{1-b-c}Co_{b}B′_{c}D_{α}, where 0.90≤a≤1.8, 0≤b≤0.5, 0≤c≤0.05, and 0<α≤2; LiₐNi_{1-b-c}Co_{b}B′_{c}O_{2-α}F′_{α}, where 0.90≤a≤1.8, 0≤b≤0.5, 0≤c≤0.05, and 0<α<2; LiₐNi_{1-b-c}Co_{b}B′_{c}O_{2-α}F′₂, where 0.90≤a≤1.8, 0≤b≤0.5, 0≤c≤0.05, and 0<α<2; LiₐNi_{1-b-c}Mn_{b}B′_{c}D_{α}, where 0.90≤a≤1.8, 0≤b≤0.5, 0≤c≤0.05, and 0<α≤2; LiₐN_{1-b-c}Mn_{b}B′_{c}O_{2-α}F′_{α}, where 0.90≤a≤1.8, 0≤b≤0.5, 0≤c≤0.05, and 0<α<2; LiₐNi_{b}E_{c}G_{d}O₂, where 0.90≤a≤1.8, 0≤b≤0.9, 0≤c≤0.5, and 0.001≤d≤0.1; LiₐNi_{b}Co_{c}Mn_{d}GeO₂, where 0.90≤a≤1.8, 0≤b≤0.9, 0≤c≤0.5, 0≤d≤0.5, and 0.001≤e≤0.1; LiₐNiG_{b}O₂, where 0.90≤a≤1.8 and 0.001≤b≤0.1; LiₐCoG_{b}O₂, where 0.90≤a≤1.8 and 0.001≤b≤0.1; LiₐMnG_{b}O₂, where 0.90≤a≤1.8 and 0.001≤b≤0.1; LiₐMn₂G_{b}O₄, where 0.90≤a≤1.8 and 0.001≤b≤0.1; QO₂; QS₂; LiQS₂; V₂O₅; LiV₂O₅; LiI′O₂; LiNiVO₄; Li_{(3-f)}J₂(PO₄)₃, where 0≤f≤2; Li_{(3-f)}Fe₂(PO₄)₃, where 0≤f≤2; and LiFePO₄.

In the above formulae, A is at least one of Ni, Co, or Mn; B′ is at least one of Al, Ni, Co, Mn, Cr, Fe, Mg, Sr, V, or a rare earth element; D is at least one of oxygen (O), fluorine (F), sulfur (S), or phosphorus (P); E is at least one of Co or Mn; F′ is at least one of F, S, or P; G is at least one of Al, Cr, Mn, Fe, Mg, lanthanum (La), cerium (Ce), strontium (Sr), or V; Q is at least one of titanium (Ti), molybdenum (Mo), or Mn; I′ is at least one of Cr, V, Fe, scandium (Sc), or yttrium (Y); and J is at least one of V, Cr, Mn, Co, Ni, or copper (Cu).

For example, the positive electrode active material may be LiCoO₂, LiMnₓO₂ₓ where x=1 or 2, LiNi₁₋ₓMnₓO₂ₓ where 0<x<1, LiNi_{1-x-y}CoₓMn_{y}O₂ where 0≤x≤0.5, 0≤y≤0.5, and 1−x−y>0.5, LiFePO₄, or the like.

The above-listed compounds having a coating layer on their surfaces may also be used, or a combination of at least one the above-listed compounds and the compound having a coating layer may be used. The coating layer may include an oxide, a hydroxide, an oxyhydroxide, an oxycarbonate, or a hydroxycarbonate of a coating element. The compounds constituting the coating layer may be amorphous or crystalline. The coating element included in the coating layer may be at least one of Mg, Al, Co, potassium (K), sodium (Na), calcium (Ca), silicon (Si), Ti, V, tin (Sn), germanium (Ge), gallium (Ga), boron (B), arsenic (As), or zirconium (Zr). The coating layer may be formed using any coating method that does not adversely affect the physical properties of the positive electrode active material (e.g., spray coating, dipping, or the like). The coating methods are understood by one of ordinary skill in the art, and thus a detailed description thereof is omitted.

The positive electrode active material composition may further include a conductive material, a filler, or the like.

The conductive material is generally added in an amount of about 1 wt % to about 30 wt %, with respect to a total weight of the positive electrode active material composition. The conductive material is not particularly limited as long as it does not cause a chemical change in the fabricated battery and has electrical conductivity. Non-limiting examples of the conductive material include: graphite such as natural graphite or artificial graphite; carbon black, acetylene black, Ketjen black, channel black, furnace black, lamp black, and thermal black; conductive fibers such as carbon fiber or metallic fiber; carbon fluoride; metallic powders such as aluminum powder and nickel powder; conductive whiskers such as zinc oxide and potassium titanate; conductive metal oxides such as titanium oxide; and polyphenylene derivatives. A combination comprising at least one of the foregoing conductive materials may also be used.

The binder is a component which facilitates binding between an active material and the conductive material and between an active material and a current collector, and is added in an amount of about 1 wt % to about 30 wt % with respect to the total weight of the positive electrode active material composition. Non-limiting examples of the binder include polyvinylidene fluoride (PVdF), polyvinylidene chloride, polybenzimidazole, polyimide, polyvinyl acetate, polyacrylonitrile, polyvinyl alcohol, carboxymethylcellulose (CMC), starch, hydroxypropylcellulose, regenerated cellulose, polyvinylpyrrolidone, polyethylene, polypropylene, polystyrene, polymethylmethacrylate, polyaniline, acrylonitrilebutadienestyrene, phenol resin, epoxy resin, polyethylene terephthalate, polytetrafluoroethylene, polyphenylenesulfide, polyamideimide, polyetherimide, polyethylenesulfone, polyamide, polyacetal, polyphenyleneoxide, polybutylene terephthalate, ethylene-propylene-diene terpolymer (EPDM), sulfonated EPDM, styrene butadiene rubber (SBR), fluorine rubber, and various copolymers. A combination comprising at least one of the foregoing binders may also be used.

The filler is a component that suppresses the expansion of a positive electrode and optionally used, and is not particularly limited as long as it does not cause a chemical change in the fabricated battery and is a fibrous material. Non-limiting examples of the filler include olefin polymers such as polyethylene and polypropylene; and fibrous materials such as glass fiber and carbon fiber.

As the solvent, at least one of N-methylpyrrolidone, acetone, or water may be used, but is not limited thereto, and any suitable solvent for a lithium battery may be used. The amount of the solvent may be in a range of, for example, about 10 parts by weight to about 100 parts by weight, with respect to 100 parts by weight of the positive electrode active material composition. When the amount of the solvent is within the above range, the formation of an active material layer is facilitated.

The amounts of the positive electrode active material, the conductive material, the filler, the binder, and the solvent in the positive electrode active material composition are not limited, and may be determined by a person of skill in the art without undue experimentation. At least one of the conductive material, the filler, the binder, and the solvent may be omitted depending on the use and configuration of a lithium battery.

For example, N-methylpyrrolidone (NMP) may be used as a solvent, PVdF or a PVdF copolymer may be used as a binder, and carbon black or acetylene black may be used as a conductive material. For example, 94 wt % of a positive electrode active material, 3 wt % of a binder, and 3 wt % of a conductive material may be mixed in a powder form, NMP may be added to the resulting mixture to prepare a slurry in which the solid content is 70 wt %, and the slurry may be coated, dried, and pressed, thereby completing the fabrication of a positive electrode.

The positive electrode current collector is generally fabricated to have a thickness of about 3 μm to about 50 μm. The positive electrode current collector is not particularly limited as long as it causes no chemical change in the fabricated battery and has high conductivity. Non-limiting examples of the positive electrode current collector include stainless steel; aluminum; nickel; titanium; sintered carbon; and copper or stainless steel that is surface-treated with carbon, nickel, titanium, or silver. In addition, the positive electrode current collector may be processed to have fine irregularities on surfaces thereof so as to enhance the adhesion of the positive electrode current collector to the positive electrode active material, and may be used in any of various forms including films, sheets, foils, nets, porous structures, foams, and non-woven fabrics.

For example, the positive electrode is manufactured by coating a positive electrode active material composition onto a positive electrode current collector and drying and pressing the resulting structure, and a positive electrode active material composition is prepared by mixing the above-described positive electrode active material and optionally, a binder and a solvent. The positive electrode active material composition is directly coated onto a metal current collector and dried, thereby completing the fabrication of a positive electrode plate. In another embodiment, the positive electrode active material composition may be cast on a separate support, and a film separated from the support may be laminated on a metal current collector, thereby completing the fabrication of a positive electrode plate.

For example, the prepared positive electrode active material composition may have a loading level of 30 milligrams per square centimeter (mg/cm²) or greater, for example, 35 mg/cm² or greater, for example, 40 mg/cm² or greater. In addition, the positive electrode active material composition may have an electrode density of 3 grams per cubic centimeter (g/cc) or greater, for example, 3.5 g/cc or greater.

In an embodiment, to achieve a high cell energy density, the prepared positive electrode active material composition may have a loading level of about 35 mg/cm² to about 50 mg/cm² and an electrode density of about 3.5 g/cc to about 4.2 g/cc.

In another embodiment, the positive electrode active material composition may be coated on opposite surfaces of the positive electrode plate to a loading level of 37 mg/cm² and an electrode density of 3.6 g/cc.

When the loading level and the electrode density of the positive electrode active material composition are within the above ranges, a battery including the positive electrode active material may exhibit a high cell energy density, e.g., 500 Wh/L or greater. For example, the battery may exhibit a cell energy density of 500 Wh/L to 900 Wh/L.

Next, a negative electrode is prepared.

For example, a negative electrode active material, a conductive material, a binder, and a solvent are mixed to prepare a negative electrode active material composition.

The negative electrode is manufactured by coating a negative electrode active material composition onto a negative electrode current collector and drying and pressing the resulting structure, and a negative electrode active material composition is prepared by mixing the negative electrode active material and optionally, a binder and a solvent.

For example, the negative electrode active material composition is directly coated onto a negative electrode current collector and dried, thereby completing the fabrication of a negative electrode. In another embodiment, the negative electrode active material composition may be cast on a separate support, and a film separated from the support may be laminated on a metal current collector, thereby completing the fabrication of a negative electrode plate.

The negative electrode active material may be, for example, a silicon compound, a silicon oxide (SiOₓ, wherein 0<x<2), or a composite of a silicon-based compound and a carbon-based material. In this regard, the silicon particles have a size (e.g., an average diameter) of less than 200 nm, for example, about 10 nm to about 150 nm. The term “size” as used herein refers to an average diameter of silicon particles when the silicon particles are spherical in shape, and refers to an average length of a major axis when the silicon particles are non-spherical in shape.

When the size of the silicon particles is within the above range, excellent lifespan characteristics may be provided, and thus when an electrolyte according to an embodiment is used, a lithium battery may exhibit further enhanced lifespan.

The carbon-based material may be at least one of crystalline carbon or amorphous carbon. The crystalline carbon may be graphite, such as natural graphite or artificial graphite that is in amorphous, plate, flake, spherical or fibrous form. The amorphous carbon may be soft carbon (carbon sintered at low temperatures), hard carbon, mesophase pitch carbide, sintered coke, or the like.

The composite of a silicon-based compound and a carbon-based material may be, for example, a composite having a structure in which silicon particles are disposed on a surface of graphite or a composite in which Si particles are disposed on a surface of and inside of the graphite. The composite may be, for example, an active material obtained by dispersing Si particles having an average diameter of about 200 nm or less, for example, about 100 nm to about 200 nm, for example, 150 nm, on graphite particles and disposing a carbon coating thereon, or an active material in which Si particles are present on and inside of graphite. These composites are available under the trade name SCN1™ (Si particles on graphite) or SCN2™ (Si particles inside of as well as on graphite). SCN1 is an active material obtained by dispersing Si particles having an average diameter of about 150 nm on graphite particles and then performing carbon coating thereon. SCN2 is an active material in which Si particles having an average diameter of about 150 nm are present on and inside graphite.

The negative electrode active material may be used in combination with another negative electrode active material suitable for a lithium battery, in addition to the above-described negative electrode active material. Examples of the negative electrode active material include Si, Sn, Al, Ge, Pb, Bi, Sb, a Si—Y′ alloy (where Y′ is at least one of an alkali metal, an alkaline earth metal, Groups 13 to 16 elements, a transition metal, a transition metal oxide, or a rare earth element, except for Si), and a Sn—Y′ alloy (where Y′ is at least one of an alkali metal, an alkaline earth metal, Groups 13 to 16 elements, a transition metal, or a rare earth element, except for Sn). Y may be at least one of magnesium (Mg), calcium (Ca), strontium (Sr), barium (Ba), radium (Ra), scandium (Sc), yttrium (Y), titanium (Ti), zirconium (Zr), hafnium (Hf), rutherfordium (Rf), vanadium (V), niobium (Nb), tantalum (Ta), dubnium (Db), chromium (Cr), molybdenum (Mo), tungsten (W), seaborgium (Sg), technetium (Tc), rhenium (Re), bohrium (Bh), iron (Fe), lead (Pb), ruthenium (Ru), osmium (Os), hassium (Hs), rhodium (Rh), iridium (Ir), palladium (Pd), platinum (Pt), copper (Cu), silver (Ag), gold (Au), zinc (Zn), cadmium (Cd), boron (B), aluminum (Al), gallium (Ga), tin (Sn), indium (In), germanium (Ge), phosphorus (P), arsenic (As), antimony (Sb), bismuth (Bi), sulfur (S), selenium (Se), tellurium (Te), or polonium (Po).

For example, the negative electrode active material may be at least one of lithium titanium oxide, vanadium oxide, or lithium vanadium oxide.

The negative electrode active material composition may further include a conductive material, a filler, or the like.

Also, in the negative electrode active material composition, the binder, the solvent, the conductive material, and the filler may be the same as those used in the positive electrode active material composition.

In the negative electrode active material composition, water may be used as a solvent. For the negative electrode active material composition may include water as a solvent, carboxymethylcellulose (CMC), styrene butadiene rubber (SBR), a binder including at least one of an acrylate-based polymer, or a methacrylate-based polymer, and a conductive material including at least one of carbon black, acetylene black, or graphite.

The amounts of the negative electrode active material, the conductive material, the binder, and the solvent may be determined by one of skill the art without undue experimentation. At least one of the conductive material, the binder, and the solvent may be omitted depending on the use and configuration of a lithium battery.

For example, 94 wt % of a negative electrode active material, 3 wt % of a binder, and 3 wt % of a conductive material may be mixed in a powder form, water may be added to the resulting mixture such that the content of solids becomes 70 wt % to thereby prepare a slurry, and the slurry may be coated, dried, and pressed, thereby completing the fabrication of a negative electrode plate.

The negative electrode current collector may be fabricated to have a thickness of about 3 μm to about 50 μm. The negative electrode current collector is not particularly limited as long as it causes no chemical change in the fabricated battery and has conductivity. Non-limiting examples of the negative electrode current collector include copper; stainless steel; aluminum; nickel; titanium; sintered carbon; copper or stainless steel that is surface-treated with carbon, nickel, titanium, or silver; and aluminum-cadmium alloys. In addition, as in the positive electrode current collector, the negative electrode current collector may be processed to have fine irregularities on at least one surface thereof so as to enhance the adhesion of the negative electrode current collector to the negative electrode active material, and may be used in any of various forms including films, sheets, foils, nets, porous structures, foams, and non-woven fabrics.

The loading level of the prepared negative electrode active material composition is set in accordance with the loading level of the positive electrode active material composition.

For example, depending on capacity/g of the negative electrode active material composition, the loading level may be 12 mg/cm² or greater, for example, 15 mg/cm² or greater. In addition, the electrode density of the negative electrode active material composition may be 1.5 g/cc or greater, for example, 1.6 g/cc or greater.

In an embodiment, to achieve a high cell energy density, the prepared negative electrode active material composition may have a loading level of about 15 mg/cm² to about 25 mg/cm² and an electrode density of about 1.6 g/cc to about 2.3 g/cc.

When the loading level and the electrode density of the negative electrode active material are within the above ranges, a battery including this negative electrode active material may exhibit a high cell energy density, i.e., 600 Wh/L or higher.

Next, a separator to be inserted between the positive electrode and the negative electrode is prepared.

The separator may be any separator suitable for use in a lithium battery. A separator having low resistance to ion transfer in an electrolyte and having an excellent electrolyte-retaining ability may be used. For example, the separator may be at least one of glass fiber, polyester, Teflon, polyethylene, polypropylene, or polytetrafluoroethylene (PTFE), each of which may be a non-woven or woven fabric. For example, a windable separator made of polyethylene, polypropylene, or the like may be used in lithium-ion batteries, and a separator having an excellent electrolyte-impregnating ability may be used in lithium-ion polymer batteries. For example, the separator may be manufactured according to the following method.

A polymer resin, a filler, and a solvent are mixed to prepare a separator composition. The separator composition may be directly coated onto an electrode and dried to thereby form a separator. In another embodiment, the separator composition may be cast on a support and dried, and then a separator film separated from the support may be laminated on an electrode, thereby completing the formation of a separator.

The polymer resin used in the formation of a separator is not particularly limited, and any material suitable for use as a binder of an electrode plate may be used. For example, the polymer resin may be at least one of a vinylidene fluoride/hexafluoropropylene copolymer, PVdF, polyacrylonitrile, or polymethylmethacrylate.

Next, the above-described electrolyte is prepared.

According to an embodiment, the electrolyte may further include, in addition to the above-described electrolyte, a non-aqueous electrolyte solution, an organic solid electrolyte, or an inorganic solid electrolyte.

Non-limiting examples of the organic solid electrolyte include at least one of a polyethylene derivative, a polyethylene oxide derivative, a polypropylene oxide derivative, a phosphoric acid ester polymer, polyester sulfide, polyvinyl alcohol, polyvinylidene fluoride, or a polymer having ionic dissociation groups.

Non-limiting examples of the inorganic solid electrolyte include at least one of Li₃N, LiI, Li₅NI₂, Li₃N—LiI—LiOH, LiSiO₄, Li₂SiS₃, Li₄SiO₄, Li₄SiO₄—LiI—LiOH, or Li₃PO₄—Li₂S—SiS₂.

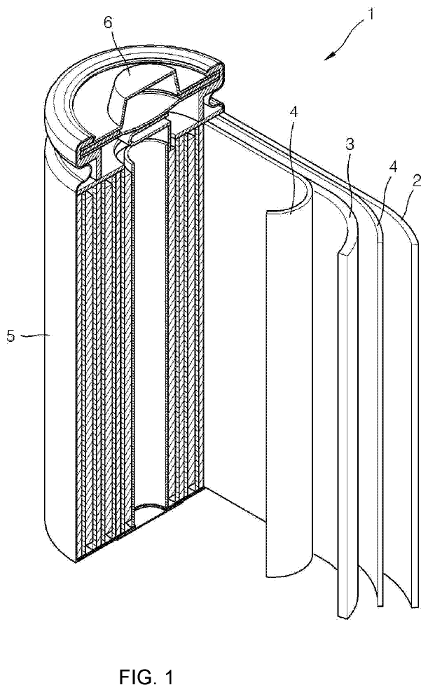

As illustrated in FIG. 1, a lithium battery 1 includes a positive electrode 3, a negative electrode 2, and a separator 4. The positive electrode 3, the negative electrode 2, and the separator 4 are wound or folded, and then accommodated in a battery case 5. Subsequently, an electrolyte is injected into the battery case 5 and the battery case 5 is sealed by a cap assembly 6 to thereby complete the manufacture of the lithium battery 1. The battery case 5 may have, for example, a cylindrical, rectangular, or thin-film form. The lithium battery 1 may be a lithium secondary battery. For example, the lithium secondary battery may be a large-scale thin-film-type battery. The lithium secondary battery may be a lithium-ion battery.

A separator may be disposed between the positive electrode and the negative electrode to form a battery assembly. A plurality of battery assemblies may be stacked in a bi-cell structure and impregnated with an electrolyte, and the resultant structure may be put into a pouch and hermetically sealed to thereby complete the manufacture of a lithium-ion polymer battery.

In addition, the battery assemblies may be stacked to form a battery pack, and such a battery pack may be used in any device requiring high capacity and high power output. For example, the battery pack may be used in notebook computers, smartphones, electric vehicles, and the like.

A lithium secondary battery according to an embodiment may exhibit excellent characteristics due to a significantly reduced DCIR increase rate thereof, compared to a general lithium secondary battery employing a Ni-rich lithium nickel composite oxide as a positive electrode active material.

An operating voltage of the lithium secondary battery including the positive electrode, the negative electrode, and the electrolyte may have, for example, a lower limit of about 2.5 volts (V) to about 2.8 and an upper limit of about 4.1 V to about 4.4 V, and the lithium secondary battery has an excellent energy density, i.e., 500 Wh/L or greater.

In addition, examples of the lithium secondary battery include, but are not limited to, power tools driven by an electric motor; electric vehicles (EVs), hybrid EVs (HEVs), and plug-in HEVs (PHEVs); electric two-wheel vehicles including E-bikes and E-scooters; electric golf carts; and power storage systems.

As used herein, the term “alkyl” refers to completely saturated, branched or unbranched (or a straight or linear) monovalent hydrocarbon group. Alkyl groups include, for example, groups having from 1 to 50 carbon atoms (C₁ to C₅₀ alkyl) unless otherwise indicated.

Non-limiting examples of the “alkyl” include methyl, ethyl, n-propyl, isopropyl, n-butyl, isobutyl, sec-butyl, n-pentyl, isopentyl, neopentyl, n-hexyl, 3-methylhexyl, 2,2-dimethylpentyl, 2,3-dimethylpentyl, and n-heptyl.

At least one hydrogen atom of the “alkyl” may be substituted with a halogen atom, a C₁-C₂₀ alkyl group substituted with a halogen atom (e.g., —CCF₃, —CHCF₂, —CH₂F, —CCl₃, or the like), a C₁-C₂₀ alkoxy group, a C₂-C₂₀ alkoxyalkyl group, a hydroxyl group, a nitro group, a cyano group, an amino group, an amidino group, hydrazine, hydrazone, a carboxyl group or a salt thereof, a sulfonyl group, a sulfamoyl group, sulfonic acid group or a salt thereof, phosphoric acid or a salt thereof, a C₁-C₂₀ alkyl group, a C₂-C₂₀ alkenyl group, a C₂-C₂₀ alkynyl group, a C₁-C₂₀ heteroalkyl group, a C₆-C₂₀ aryl group, a C₆-C₂₀ arylalkyl group, a C₆-C₂₀ heteroaryl group, a C₇-C₂o heteroarylakyl group, a C₆-C₂₀ heteroaryloxy group, a C₆-C₂₀ heteroaryloxyalkyl group, or a C₆-C₂₀ heteroarylalkyl group.

The term “halogen” as used herein includes fluorine, bromine, chloride, iodine, and astatine.

The term “alkoxy” refers to an alkyl group linked to an oxygen (alkyl-O—). The alkoxy group may be, for example, a methoxy group, an ethoxy group, a 2-propoxy group, a butoxy group, a t-butoxy group, a pentyloxy group, a hexyloxy group, or the like. At least one hydrogen atom of the alkoxy group may be substituted with a substituent as described above.

The term “alkenyl” refers to a branched or non-branched, monovalent hydrocarbon having at least one carbon-carbon double bond. Non-limiting examples of the alkenyl group include vinyl, allyl (H₂C═CH—CH₂—), butenyl, propenyl, and isobutenyl, and at least one hydrogen atom of the alkenyl group may be substituted with the same substituent as in the above-described alkyl group.

“Vinyl” group includes any group having terminal unsaturation (—CH₂═CH₂), including acrylate groups (—OC(O)CH═CH₂) and methacrylate (—OC(O)(CH₃)═CH₂) groups.

The term “alkynyl” refers to branched or non-branched divalent hydrocarbon having at least one carbon-carbon triple bond. Non-limiting examples of the “alkynyl” include ethynyl, butynyl, isobutynyl, and isopropynyl.

At least one hydrogen atom of the “alkynyl” may be substituted with a substituent as described above.

The term “aryl” refers to a monovalent group having an aromatic ring, and includes monocyclic and polycyclic hydrocarbons wherein the additional ring(s) of the polycyclic hydrocarbon may be aromatic or nonaromatic. Non-limiting examples of the aryl group include phenyl, naphthyl, and tetrahydronaphthyl. In addition, at least one hydrogen atom of the “aryl” may be substituted a substituent as described above.

The term “heteroaryl” refers to means a monovalent carbocyclic ring group that includes one or more aromatic rings, in which at least one ring member (e.g., one, two or three ring members) is a heteroatom independently N, O, P, or S, with the remainder of the atoms being carbon. In a C3 to C30 heteroaryl, the total number of ring carbon atoms ranges from 3 to 30, with remaining ring atoms being heteroatoms. S or N may be present in various oxidized forms.

Examples of the heteroaryl group include thienyl, furyl, pyrrolyl, imidazolyl, pyrazolyl, thiazolyl, isothiazolyl, 1,2,3-oxadiazolyl, 1,2,4-oxadiazolyl, 1,2,5-oxadiazolyl, 1,3,4-oxadiaxolyl, 1,2,3-thiadiazolyl, 1,2,4-thiadiazolyl, 1,2,5-thiadiazolyl, 1,3,4-thiadiazolyl, isothiazol-3-yl, isothiazol-4-yl, isothiazol-5-yl, oxazol-2-yl, oxazol-4-yl, oxazol-5-yl, isoxazol-3-yl, isoxazol-4-yl, isoxazol-5-yl, 1,2,4-triazol-3-yl, 1,2,4-triazol-5-yl, 1,2,3-triazol-4-yl, 1,2,3-triazol-5-yl, tetrazolyl, pyrid-2-yl, pyrid-3-yl, 2-pyrazin-2-yl, pyrazin-4-yl, pyrazin-5-yl, 2-pyrimidin-2-yl, 4-pyrimidin-2-yl, and 5-pyrimidin-2-yl.

The term “heteroaryl” includes a heteroaromatic ring selectively fused to at least one aryl, cycloaliphatic or heterocyclic group.

“Alkenylene” means a straight or branched chain, divalent hydrocarbon group having at least one carbon-carbon double bond (e.g., ethenylene (—HC═CH—)).

“Alkynylene” means a straight or branched chain divalent aliphatic hydrocarbon that has one or more unsaturated carbon-carbon bonds, at least one of which is a triple bond (e.g., ethynylene).

“Acid anhydride” refers to a group having two acyl groups joined together by an oxygen atom.

“Hydrocarbon” means an organic compound having at least one carbon atom and at least one hydrogen atom, optionally substituted with one or more substituents where indicated.

Hereinafter, the present disclosure will be described in further detail with reference to the following examples and comparative examples. However, these examples are provided for illustrative purposes only and are not intended to limit the scope of the present disclosure.

### Examples

### Examples 1 to 6 and Comparative Examples 1 to 14

Lithium batteries were manufactured in accordance with the configurations shown in Tables 2 to 6 below. Each configuration was manufactured in detail as follows.

(Manufacture of Positive Electrode 1)

LiNi_{0.88}Co_{0.10}Mn_{0.02}O₂ as a positive electrode active material, carbon black as a conductive material, and PVdF as a binder were added into N-methylpyrrolidone (NMP) in a weight ratio of 97.7:1:1.1 and mixed. The resulting solution was coated onto opposite surfaces of Al foil having a thickness of 15 micrometers (μm) by dispersion to an area of 33 milligrams per square centimeter (mg/cm²) per surface, dried, and pressed, thereby completing the manufacture of positive electrode 1 having an electrode density of 3.6 grams per cubic centimeter (g/cc).

### (Manufacture of Negative Electrode 1)

A pitch-coated, silicon-graphite (Si:Gr) composite (weight ratio of silicon to graphite of 14.9:81.1) as a negative electrode active material and an AG binder (commercially available from Sumitomo Seika) were added to NMP in a weight ratio of 96:4 and mixed, and the resulting solution was coated onto opposite surfaces of Cu foil having a thickness of 8 μm by dispersion to an area of 17.6 mg/cm² per surface, dried, and pressed, thereby completing the manufacture of negative electrode 1 having an electrode density of 1.65 g/cc.

### (Manufacture of Negative Electrode 2)

Pitch-free Si:Gr (weight ratio of 14.9:81.1) as a negative electrode active material and an AG binder were added to NMP in a weight ratio of 96:4 and mixed, and the resulting solution was coated onto opposite surfaces of Cu foil having a thickness of 8 μm by dispersion to an area of 17.6 mg/cm² per surface, dried, and pressed, thereby completing the manufacture of negative electrode 2 having an electrode density of 1.65 g/cc.

### (Manufacture of Negative Electrode 3)

MC09:MC21:MC10 as a negative electrode active material (weight ratio of 20:70:10, commercially available from Mitsubishi) and BM480B/BSH12 as a binder (weight ratio of 1.5:1.0, BM480B is manufactured by and available from ZEON, and BSH12 is manufactured by and available from Dai-ichi Kogyo Seiyaku) were added to NMP in a weight ratio of 96:4 and mixed. The resulting solution was coated onto opposite surfaces of Cu foil having a thickness of 8 μm by dispersion to an area of 17.6 mg/cm² per surface, dried, and pressed, thereby completing the manufacture of negative electrode 3 having an electrode density of 1.65 g/cc.

### (Preparation of Electrolyte)

As solvent 1, 1.15M LiPF₆ and FEC/EC/EMC/DMC (volume ratio: 3/10/47/40) was used.

As solvent 2, 1 wt % of VC, 0.2 wt % of LiFSI, and 2 wt % of TMP were added to 1.3M LiPF₆ and FEC/EC/EMC/DMC (volume ratio: 7/10/13/70).

As solvent 3, 1 wt % of VC, 0.2 wt % of LiFSI, and 2 wt % of TMP were added to 1.3M LiPF₆ and FEC/EC/EMC/DMC (volume ratio: 5/10/15/70).

To each of solvents 1 to 3, additives were added as listed in Tables 2 to 6 to prepare the respective electrolyte. The chemical structure of each additive is shown in Table 1.

TABLE 1

| | | Chemical |
|---|---|---|
| Abbreviation | Chemical Name | structure |
| MMSN | Methyl methaneuftonate | 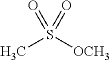 |
| EMS | Ethylmethane sulfone | 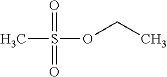 |
| PhpTs | Phenyl p-Toluenesulfonate | 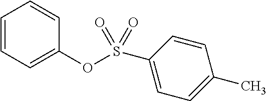 [SMILES: Cc1ccc(S(=O)(=O)Oc2ccccc2)cc1] |
| PMSN | Phenyl methanesulfonate | 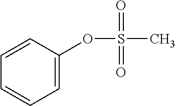 |
| EVS | Ethyl vinyl sulfone | 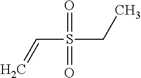 |
| PTSN | Propargyl p-toluenesulfonate | 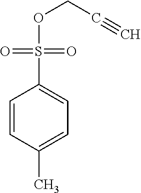 |
| AMS | Allyl methyl sulfone | 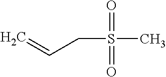 |
| TMP | Trimethylphosphate | 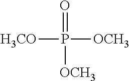 |
| EMSF | Ethyl methyl sulfone | 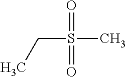 |
| DPhS | Diphenyl sulfone | 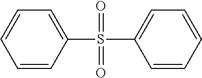 [SMILES: O=S(=O)(c1ccccc1)c1ccccc1] |

### (Assembly of Lithium Battery)

A separator made of polypropylene and having a thickness of 16 μm was disposed between the positive electrode and each negative electrode, and the electrolyte was injected into the separator for the manufacture of a lithium battery.

### Evaluation Example 1: Evaluation of Gas Generation Amount and Direct Current Internal Resistance (DCIR) Characteristics

Each of the lithium batteries manufactured according to Examples 1 to 4 and Comparative Examples 1 to 9 was charged at a constant current of 0.2 C rate at 25° C. until the voltage reached 3.6 volts (V) (vs. Li), and discharged at a constant current of 0.2 rate until the voltage reached 2.8 V (vs. Li) (1^{st} cycle of formation operation). Subsequently, each lithium battery was charged at a constant current of 0.2 C rate until the voltage reached 4.25 V (vs. Li). Thereafter, each lithium battery was discharged at a constant current of 0.2 rate until the voltage reached 2.8 V (vs. Li) (2^{nd} cycle of formation operation). Third, each lithium battery was charged at a constant current of 0.5 C rate until the voltage reached 4.25 V (vs. Li) and then, while maintaining a constant voltage of 4.25 V, the charging process was cut off at a current of 0.05 C rate. Subsequently, each lithium battery was discharged at a constant current of 0.2 rate until the voltage reached 2.8 V (vs. Li). For the 4th cycle, the 3^{rd} formation operation was repeated (0.5 C charging/0.2 C discharging). Lastly, each lithium battery was charged at a constant current of 0.2 C rate until the voltage reached 4.25 V and, while maintaining a constant voltage of 4.25 V, the charging process was cut off at a current of 0.05 C rate.

The expression “1C charging” means that battery capacity (mAh) is reached by charging for 1 hour. Likewise, the expression “1C discharging” means that the battery capacity (mAh) is completely consumed by discharging for 1 hour.

The lithium batteries having undergone the formation operations were maintained at 60° C. for 10 days to evaluate gas generation amounts and DCIR characteristics, and the results are shown in Tables 2 and 3.

TABLE 2

| | | | | | Gas generation | | | |
|---|---|---|---|---|---|---|---|---|
| | | | | | amount | 0 D | 10 D | |
| | Electrolyte | | Positive | Negative | (@60° C. 10 D) | DCIR | DCIR | ΔDCIR |
| | Solvent | Additive | electrode | electrode | Relative value (%)] | (mΩ) | (mΩ) | (%) |
| Example 1 | Solvent 1 | 0.5 wt % MMSN | Positive | Negative | 0.67 | 131 | 127 | 97 |
| | | | electrode 1 | electrode 1 | | | | |
| Example 2 | Solvent 1 | 0.5 wt % EMS | Positive | Negative | 0.75 | 133 | 129 | 97 |
| | | | electrode 1 | electrode 1 | | | | |
| Comparative | Solvent 1 | — | Positive | Negative | 0.78 | 130 | 130 | 100 |
| Example 1 | | | electrode 1 | electrode 1 | | | | |
| Comparative | Solvent 1 | 0.5 wt % PhpTs | Positive | Negative | 0.72 | 137 | 140 | 108 |
| Example 2 | | | electrode 1 | electrode 1 | | | | |
| Comparative | Solvent 1 | 0.5 wt % PMSN | Positive | Negative | 0.78 | 130 | 132 | 101 |
| Example 3 | | | electrode 1 | electrode 1 | | | | |
| Example 3 | Solvent 1 | 1 wt % MMSN | Positive | Negative | 0.77 | 124 | 132 | 107 |
| | | | electrode 1 | electrode 2 | | | | |
| Comparative | Solvent 1 | 0.3 wt % PTSN + | Positive | Negative | 0.77 | 122 | 133 | 109 |
| Example 4 | | 0.3 wt % MMSN | electrode 1 | electrode 2 | | | | |
| Comparative | Solvent 1 | 1 wt % sulfolane | Positive | Negative | 0.82 | 126 | 138 | 109 |
| Example 5 | | | electrode 1 | electrode 2 | | | | |

TABLE 3

| | | | | | Gas generation | | | |
|---|---|---|---|---|---|---|---|---|
| | | | | | amount | 0 D | 10 D | |
| | Electrolyte | | Positive | Negative | (@60° C. 10 D) | DCIR | DCIR | ΔDCIR |
| | Solvent | Additive | electrode | electrode | [Relative value (%)] | (mΩ) | (mΩ) | (%) |
| Example 4 | Solvent 2 | 0.3 wt % EVS + | Positive | Negative | 0.64 | 142 | 171 | 121 |
| | | 0.3 wt % MMSN | electrode 1 | electrode 2 | | | | |
| Comparative | Solvent 2 | — | Positive | Negative | 0.90 | 148 | 189 | 127 |
| Example 6 | | | electrode 1 | electrode 2 | | | | |
| Comparative | Solvent 2 | 0.6 wt % EVS | Positive | Negative | 0.70 | 148 | 192 | 130 |
| Example 7 | | | electrode 1 | electrode 2 | | | | |
| Comparative | Solvent 2 | 0.3 wt % EVS + | Positive | Negative | 0.79 | 150 | 198 | 132 |
| Example 8 | | 0.3 wt % AMS | electrode 1 | electrode 2 | | | | |
| Comparative | Solvent 2 | 0.3 wt % PTSN + | Positive | Negative | 0.73 | 148 | 192 | 129 |
| Example 9 | | 0.3 wt % MMSN | electrode 1 | electrode 2 | | | | |

Referring to Table 2, the lithium batteries of Examples 1 and 2 exhibited gas generation amounts that were lower than or equivalent to those of the lithium batteries of Comparative Examples 1 to 3, which included different additives under the same conditions. Moreover, the lithium batteries of Examples 1 and 2 exhibited significantly reduced DCIR increase rates, which indicates excellent stability. In particular, the lithium secondary batteries of Examples 1 and 2 exhibited substantially lowered DCIRs when stored at a high temperature, i.e., at 60° C. for 10 days.

In addition, referring to Table 2, the lithium battery of Example 3 exhibited significantly reduced gas generation and DCIR increase rate, which indicates excellent stability, as compared to the lithium batteries of Comparative Examples 4 and 5, which included different additives under the same conditions.

In addition, referring to Table 3, the lithium battery of Example 4 exhibited significantly reduced gas generation and DCIR increase rate, which indicates excellent stability, as compared to the lithium batteries of Comparative Examples 6 to 9, which included different additives under the same conditions.

Evaluation Example 2: Evaluation of Gas Generation Amount and DCIR Characteristics

Each of the lithium batteries of Examples 5 and 6 and Comparative Examples 10 and 11 was subjected to a formation operation in the same manner as in Evaluation Example 1, followed by 100 cycles of charging and discharging at 45° C. under conditions of a charging/discharging current of 1C/1C, an operating voltage of 2.8 V to 4.25 V, and cut-off at CC-CV 1/10C, For each battery the capacity retention rate and DCIR of each lithium battery were measured. The results are listed in Table 4 below.

TABLE 4

| | | | | | Capacity | | | |
|---|---|---|---|---|---|---|---|---|
| | | | | | retention rate | 0 D | 10 D | |
| | Electrolyte | | Positive | Negative | (%, @45° C., | DCIR | DCIR | ΔDCIR |
| | Solvent | Additive | electrode | electrode | 100CY) | (mΩ) | (mΩ) | (%) |
| Example 5 | Solvent 3 | 0.3 wt % EVS + | Positive | Negative | 91.6 | 144 | 150 | 104 |
| | | 0.3 wt % MMSN | electrode 1 | electrode 2 | | | | |
| Comparative | Solvent 3 | 0.3 wt % PTSN + | Positive | Negative | 91.6 | 149 | 161 | 108 |
| Example10 | | 0.3 wt % MMSN | electrode 1 | electrode 2 | | | | |
| Example 6 | Solvent 3 | 0.3 wt % EVS + | Positive | Negative | 74.6 | 144 | 182 | 126 |
| | | 0.3 wt % MMSN | electrode 1 | electrode 2 | | | | |
| Comparative | Solvent 3 | 0.3 wt % PTSN + | Positive | Negative | 74.0 | 145 | 195 | 134 |
| Example 11 | | 0.3 wt % MMSN | electrode 1 | electrode 2 | | | | |

Referring to Table 4, the lithium batteries of Examples 5 and 6 exhibited substantially the same or a longer lifespan, and significantly reduced DCIR increase rates, compared to the lithium batteries of Comparative Examples 10 and 11, which included different additives under the same conditions. Accordingly, the lithium batteries of Examples 5 and 6 exhibit improved stability.

### Evaluation Example 3: Evaluation of Lifespan and DCIR Characteristics

Each of the lithium batteries of Comparative Examples 12 to 14 was charged and discharged in the same manner as in Evaluation Example 1, except that the operating voltage was adjusted to 2.8 V to 4.3 V. For each battery, gas generation amount and DCIR were measured, and the results are listed in Table 5 below.

TABLE 5

| | | | | | Gas generation | 0 D | 10 D | |
|---|---|---|---|---|---|---|---|---|
| | Electrolyte | | Positive | Negative | amount | DCIR | DCIR | ΔDCIR |
| | Solvent | Additive | electrode | electrode | (@60° C., 10 D) | (mΩ) | (mΩ) | (%) |
| Comparative | Solvent 1 | 1.5 wt % VC + | Positive | Negative | 0.62 | 191 | 219 | 114 |
| Example 12 | | 1 wt % TMP | electrode 1 | electrode 3 | | | | |
| Comparative | Solvent 1 | 1.5 wt % VC + | Positive | Negative | 0.66 | 186 | 228 | 122 |
| Example 13 | | 1 wt % EMSF | electrode 1 | electrode 3 | | | | |
| Comparative | Solvent 1 | 1.5 wt % VC + | Positive | Negative | 0.59 | 191 | 224 | 117 |
| Example 14 | | 1 wt % DPhS | electrode 1 | electrode 3 | | | | |

Referring to Table 5, when a sulfone compound was used instead of the unsaturated sulfone compound, the gas generation amount and/or the DCIR increase rate were increased as compared to the case of using TMP, which is an additive generally used in lithium batteries.

As is apparent from the foregoing description, use of an electrolyte including the alkyl sulfonate compound results in suppression of a side reaction of a lithium battery, and the resulting lithium battery exhibits reduced gas generation and enhanced lifespan characteristics. In addition, an electrolyte that includes the alkyl sulfonate compound and the unsaturated sulfone compound a side reaction of a lithium battery is suppressed and the lithium battery exhibits reduced gas generation and enhanced lifespan characteristics.

It should be understood that embodiments described herein should be considered in a descriptive sense only and not for purposes of limitation. Descriptions of features or aspects within each embodiment should be considered as available for other similar features or aspects in other embodiments.

While an embodiment has been described with reference to the figures, it will be understood by those of ordinary skill in the art that various changes in form and details may be made therein without departing from the spirit and scope as defined by the following claims.

What is claimed is:

1. An electrolyte comprising:
a lithium salt;
a non-aqueous solvent;
an alkyl sulfonate compound represented by Formula 2; and
an unsaturated sulfone compound represented by Formula 3 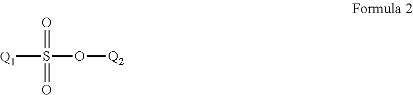
wherein, in Formula 2, Q₁ and Q₂ are each independently a substituted or unsubstituted C₁-C₂₀ alkyl group, and 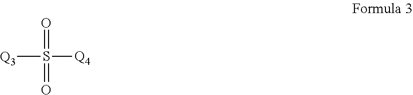
wherein, in Formula 3, Q₃ and Q₄ are each independently a group represented by -(L₁)-(R₁), a vinyl group, an allyl group, or a substituted or unsubstituted C₁-C₂₀ alkyl group, and at least one of Q₃ or Q₄ is a group represented by -(L₁)-(R₁), a vinyl group, or an allyl group,
L₁ is a substituted or unsubstituted C₂-C₂₀ alkenylene group or a substituted or unsubstituted C₂-C₂₀ alkynylene group, and
R₁ is hydrogen or a substituted or unsubstituted C₂-C₂₀ alkyl group.

2. The electrolyte of claim 1, wherein the alkyl sulfonate compound is present in a range of about 0.001 parts by weight to about 3 parts by weight with respect to 100 parts by weight of the electrolyte.

3. The electrolyte of claim 1, wherein the unsaturated sulfone compound is present in a range of about 0.001 parts by weight to about 3 parts by weight with respect to 100 parts by weight of the electrolyte.

4. The electrolyte of claim 1, wherein a sum of an amount of the alkyl sulfonate compound and an amount of the unsaturated sulfone compound is in a range of about 0.001 parts by weight to about 5 parts by weight with respect to 100 parts by weight of the electrolyte.

5. The electrolyte of claim 1, wherein
Q₁ and Q₂ are each independently a methyl group, an ethyl group, a propyl group, an isopropyl group, a butyl group, a sec-butyl group, a tert-butyl group, or an isobutyl group; or
a methyl group, an ethyl group, a propyl group, an isopropyl group, a butyl group, a sec-butyl group, a tert-butyl group, or an isobutyl group, each of which is substituted with at least one of deuterium, —F, —Cl, —Br, —I, a cyano group, a nitro group, a hydroxyl group, a methyl group, an ethyl group, or a propyl group, and
Q₃ and Q₄ are each independently a vinyl group, an allyl group, a methyl group, an ethyl group, a propyl group, an isopropyl group, a butyl group, a sec-butyl group, a tert-butyl group, or an isobutyl group; or
a methyl group, an ethyl group, a propyl group, an isopropyl group, a butyl group, a sec-butyl group, a tert-butyl group, or an isobutyl group, each of which is substituted with at least one of deuterium, —F, —Cl, —Br, —I, a cyano group, a nitro group, a hydroxyl group, a methyl group, an ethyl group, and a propyl group, and
wherein at least one of Q₃ and Q₄ is a vinyl group or an allyl group.

6. The electrolyte of claim 1, wherein the alkyl sulfonate compound is at least one of compound 1 or compound 2

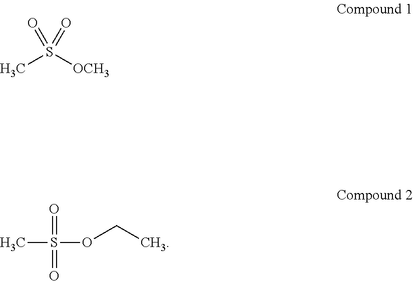

7. The electrolyte of claim 1, wherein the unsaturated sulfone compound is compound 3

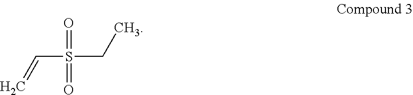

8. The electrolyte of claim 1, wherein the electrolyte does not comprise an aromatic hydrocarbon compound or an aromatic hydrocarbon compound substituted with a halogen.

9. The electrolyte of claim 1, wherein the lithium salt comprises at least one of LiPF₆, LiBF₄, LiCF₃SO₃, Li(CF₃SO₂)₂N, LiC₂F₅SO₃, Li(FSO₂)₂N, LiC₄F₉SO₃, LiN(SO₂CF₂CF₃)₂, a compound represented by Formula 22, a compound represented by Formula 23, a compound represented by Formula 24, or a compound represented by Formula 25:

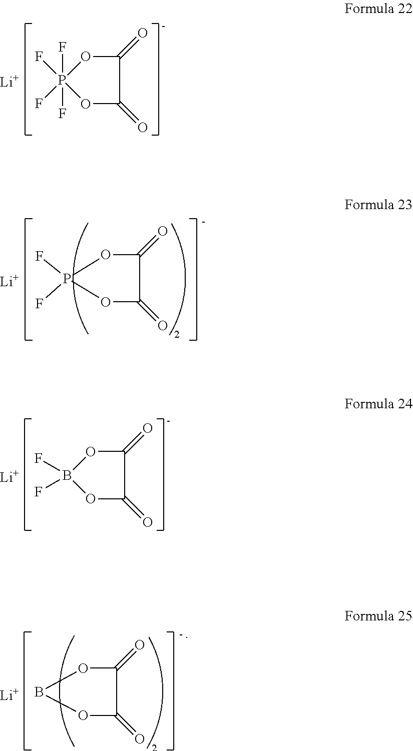

10. The electrolyte of claim 1, wherein an amount of the lithium salt in the electrolyte is in a range of about 1.1 moles per liter to about 2.5 moles per liter.

11. The electrolyte of claim 1, wherein the non-aqueous solvent comprises at least one of dimethyl carbonate, diethyl carbonate, ethyl methyl carbonate, dipropyl carbonate, methyl propyl carbonate, ethyl propyl carbonate, methyl ethyl carbonate, ethylene carbonate, propylene carbonate, butylene carbonate, methyl propionate, ethyl propionate, propyl propionate, or tetraethylene glycol dimethyl ether.

12. The electrolyte of claim 1, wherein the electrolyte further comprises at least one of fluoro-ethylene carbonate, vinylene carbonate, vinyl ethylene carbonate, maleic anhydride, succinic anhydride, a phosphorus-containing compound, or a sulfur-containing compound,
wherein the phosphorus-containing compound comprises at least one of a phosphine compound, a phosphate compound, or a phosphite compound, and
the sulfur-containing compound comprises at least one of a sulfone compound, a sulfonate compound, a sultone compound, or a disulfonate compound.

13. The electrolyte of claim 12, wherein an amount of each of fluoro-ethylene carbonate, vinylene carbonate, vinyl ethylene carbonate, maleic anhydride, succinic anhydride, the phosphorus-containing compound, or the sulfur-containing compound is in a range of about 0.1 parts by weight to about 2 parts by weight, with respect to 100 parts by weight of the electrolyte.

14. A lithium battery comprising:
a positive electrode;
a negative electrode; and
an electrolyte disposed between the positive electrode and the negative electrode,
wherein the positive electrode comprises a positive electrode active material represented by Formula 1A, and
the electrolyte comprises a lithium salt, a non-aqueous solvent, and an alkyl sulfonate compound represented by Formula 2:
LiₓNi_{y}M_{1-y}O_{2-z}A_{z} Formula 1A
wherein, in Formula 1A, 0.9≤x≤1.2, 0.88≤y≤0.98, and 0≤z≤0.2,
M is at least one of aluminum, magnesium, manganese, cobalt, iron, chromium, vanadium, titanium, copper, boron, calcium, zinc, zirconium, niobium, molybdenum, strontium, antimony, tungsten, or bismuth; and
A is an element having an oxidation number of −1, −2, or −3; 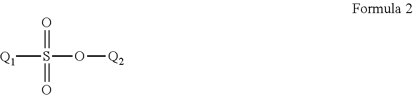
wherein, in Formula 2, Q₁ and Q₂ are each independently a substituted or unsubstituted C₁-C₂₀ alkyl group.

15. The lithium battery of claim 14, wherein an amount of the alkyl sulfonate compound is in a range of about 0.005 parts by weight to about 5 parts by weight with respect to 100 parts by weight of the electrolyte.

16. The lithium battery of claim 14, wherein Q₁ and Q₂ are each independently a methyl group, an ethyl group, a propyl group, an isopropyl group, a butyl group, a sec-butyl group, a tert-butyl group, or an isobutyl group; or
a methyl group, an ethyl group, a propyl group, an isopropyl group, a butyl group, a sec-butyl group, a tert-butyl group, or an isobutyl group, each substituted with at least one of deuterium, —F, —Cl, —Br, —I, a cyano group, a nitro group, a hydroxyl group, a methyl group, an ethyl group, or a propyl group.

17. The lithium battery of claim 14, wherein the alkyl sulfonate compound is at least one of compounds 1 or 2

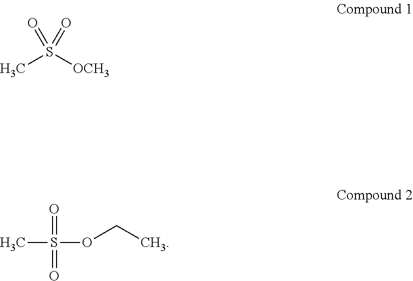

18. The lithium battery of claim 14, wherein the lithium salt comprises at least one of LiPF₆, LiBF₄, LiCF₃SO₃, Li(CF₃SO₂)₂N, LiC₂F₅SO₃, Li(FSO₂)₂N, LiC₄F₉SO₃, or LiN(SO₂CF₂CF₃)₂, a compound represented by Formula 22, a compound represented by Formula 23, a compound represented by Formula 24, or a compound represented by Formula 25

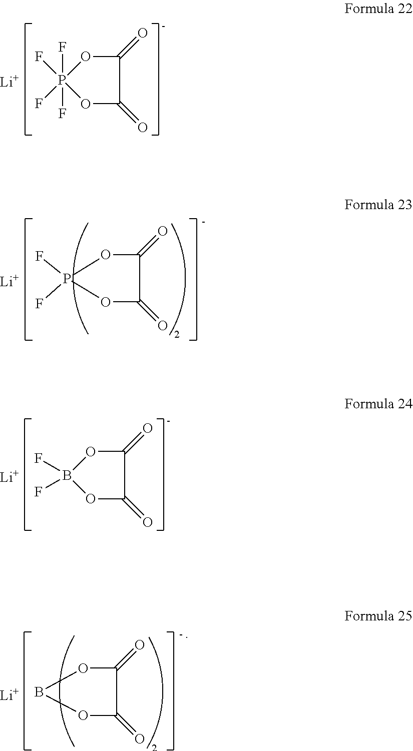

19. The lithium battery of claim 14, wherein the non-aqueous solvent comprises at least one of dimethyl carbonate, diethyl carbonate, ethyl methyl carbonate, dipropyl carbonate, methyl propyl carbonate, ethyl propyl carbonate, methyl ethyl carbonate, ethylene carbonate, propylene carbonate, butylene carbonate, methyl propionate, ethyl propionate, propyl propionate, or tetraethylene glycol dimethyl ether.

20. The lithium battery of claim 14, wherein the electrolyte further comprises at least one of fluoro-ethylene carbonate, vinylene carbonate, vinyl ethylene carbonate, maleic anhydride, succinic anhydride, a phosphorus (containing compound, or a sulfur containing compound,
wherein the phosphorus-containing compound comprises at least one of a phosphine compound, a phosphate compound, or a phosphite compound, and
the sulfur-containing compound comprises at least one of a sulfone compound, a sulfonate compound, a sultone compound, or a disulfonate compound.

21. The lithium battery of claim 14, wherein the positive electrode active material is represented by Formula 10 or Formula 20:
LiₓNi_{y′}Co_{1-y′-z′}Al_{z′}O₂ Formula 10
LiₓNi_{y′}Co_{1-y′-z′}Mn_{z′}O₂ Formula 20
wherein, in Formula 10 and Formula 20, 0.9≤x′≤1.2, 0.88≤y′≤0.98, 0<z′<0.1, and 0<1-y′-z′<0.2.

22. The lithium battery of claim 14, wherein the positive electrode comprises at least one of Li_{1.02}Ni_{0.88}Co_{0.08}Mn_{0.04}O₂, Li_{1.02}Ni_{0.88}Co_{0.10}Mn_{0.02}O₂, Li_{1.02}Ni_{0.91}Co_{0.06}Mn_{0.03}O₂, LiNi_{0.94}Co_{0.04}Mn_{0.02}O₂, Li_{1.02}Ni_{0.88}Co_{0.08}Al_{0.04}O₂, Li_{1.02}Ni_{0.88}Co_{0.10}Al_{0.02}O₂, Li_{1.02}Ni_{0.91}Co_{0.06}Al_{0.03}O₂, or LiNi_{0.94}Co_{0.04}Al_{0.02}O₂.

23. The lithium battery of claim 14, wherein the negative electrode comprises a negative electrode active material,
wherein the negative electrode active material comprises at least one of a silicon compound, a carbon compound, a composite of a silicon compound and a carbon compound, or a silicon oxide.

24. A lithium battery comprising:
a positive electrode;
a negative electrode; and
the electrolyte according to claim 1 disposed between the positive electrode and the negative electrode,
wherein the positive electrode comprises a positive electrode active material represented by Formula 1B
LiₚNi_{q}M_{1-q}O₂₋ᵣAᵣ Formula 1B
wherein, in Formula 1B, 0.9≤p≤1.2, 0.7≤q≤0.98, and 0≤r<0.2;
M is at least one of aluminum, magnesium, manganese, cobalt, iron, chromium, vanadium, titanium, copper, boron, calcium, zinc, zirconium, niobium, molybdenum, strontium, antimony, tungsten, or bismuth; and
A is an element having an oxidation number of −1, −2, or −3.

25. The lithium battery of claim 24, wherein, in Formula 1B, 0.8≤q≤0.98.

26. The lithium battery of claim 24, wherein the positive electrode active material is represented by at least one of Formula 30 or Formula 40
Li_{p′}Ni_{q′}Co_{1-q′-r′}Al_{r′}O₂ Formula 30
Li_{p′}Ni_{q′}Co_{1-q′-r′}Mn_{r′}O₂ Formula 40
wherein, in Formula 30 and Formula 40, 0.9≤p′≤1.2, 0.88≤q≤0.98, 0<r′<0.1, and 0<1−q′−r′<0.2.

27. The lithium battery of claim 24, wherein the positive electrode comprises at least one of Li_{1.02}Ni_{0.80}Co_{0.15}Mn_{0.05}O₂, Li_{1.02}Ni_{0.85}Co_{0.1}Mn_{0.05}O₂, Li_{1.02}Ni_{0.88}Co_{0.08}Mn_{0.04}O₂, Li_{1.02}Ni_{0.88}Co_{0.10}Mn_{0.02}O₂, Li_{1.02}Ni_{0.91}Co_{0.06}Mn_{0.03}O₂, LiNi_{0.94}Co_{0.04}Mn_{0.02}O₂, Li_{1.02}Ni_{0.80}Co_{0.15}Al_{0.05}O₂, Li_{1.02}Ni_{0.85}Co_{0.1}Al_{0.05}O₂, Li_{1.02}Ni_{0.88}Co_{0.08}Al_{0.04}O₂, Li_{1.02}Ni_{0.88}Co_{0.10}Al_{0.02}O₂, Li_{1.02}Ni_{0.91}Co_{0.06}Al_{0.03}O₂, or LiNi_{0.94}Co_{0.04}Al_{0.02}O₂.

28. The lithium battery of claim 24, wherein the negative electrode comprises a negative electrode active material,
wherein the negative electrode active material comprises at least one of a silicon compound, a carbon compound, a composite of a silicon compound and a carbon compound, or a silicon oxide.

29. The lithium battery of claim 24, wherein the lithium battery has a capacity retention rate of 80% or greater after 100 cycles of charge and discharge at 25° C.

30. The lithium battery of claim 24, wherein the lithium battery has a direct current internal resistance increase rate of 130 percent or less after 300 cycles of charge and discharge at 25° C.
